(19) United States
(12) Patent Application Publication
(10) Pub. No.: US 2013/0059311 A1
(43) Pub. Date: Mar. 7, 2013

(54) METHOD FOR THE IDENTIFICATION OF MEMORY MODULATING COMPOUNDS BY ASSESSING KIBRA EXPRESSION

(71) Applicants: Armin Schneider, Heidelberg (DE); Robert Spoelgen, Frankfurt am Main (DE); Tanjew Dittgen, Ulm (DE); Claudia Pitzer, Rauenberg (DE); Christian Plaas, Heidelberg (DE)

(72) Inventors: Armin Schneider, Heidelberg (DE); Robert Spoelgen, Frankfurt am Main (DE); Tanjew Dittgen, Ulm (DE); Claudia Pitzer, Rauenberg (DE); Christian Plaas, Heidelberg (DE)

(73) Assignee: SYGNIS BIOSCIENCE GMBH & CO KG, Heidelberg (DE)

(21) Appl. No.: 13/577,547

(22) PCT Filed: Feb. 8, 2011
(86) PCT No.: PCT/EP11/00581

§ 371 Date: Nov. 16, 2012

(30) Foreign Application Priority Data
Feb. 8, 2010 (EP) 10 001 267.3

(51) Int. Cl. C12Q 1/68; G01N 21/64; G01N 21/76; C12N 15/85; C12N 5/10

(52) U.S. Cl. 435/6.13; 435/320.1; 435/369; 435/353; 435/368

(57) ABSTRACT

Provided are cell based methods to identify memory modulating compounds based on the assessment of KIBRA expression. These methods are also suitable to uncover memory related pathways. The methods comprise the contacting of test compounds with cultured cells and the subsequent assessment of modulation of the KIBRA expression within the cells.

The present invention provides novel in vitro or cell based methods to identify memory modulating compounds based on the assessment of the expression of KIBRA gene product. These methods are also suitable to uncover memory related pathways or to test the relevance of memory modulating strategies. The provision describes assays which meet the needs in pharmaceutical research to find new treatment options for multiple neurological conditions where learning and memory is impaired. The technical specifications are described in here and exemplified below.

The protein KIBRA (for KIdney BRAin protein, also known as WWC1) has come into the focus of the neuroscience field since human genetic evidence demonstrates that a KIBRA allele is associated with memory performance and cognition in healthy subjects. It was found that carriers of the KIBRA (rs17070145) T allele performed better on multiple episodic memory tasks than those homozygous for the C allele at rs17070145 (Papassotiropoulos et al. Science 2006, 314:475; WO 2007/120955). Furthermore, stronger brain activation in key areas of learning and memory was observed in subjects carrying the C allele in comparison to T allele carriers during a memory task. The activation was measured during episodic memory task, using functional magnetic resonance imaging and suggests that non-carriers of the T allele need more activation in memory related brain regions to reach the same retrieval performance as the T allele carriers.

The association of KIBRA polymorphism with memory performance was confirmed in various studies in healthy subjects and in Alzheimer's disease patients (Schaper et al. Neurobiol Aging 2008, 29:1123; Almeida et al. J Cell Mol Med. 2008, 12:1672; Nacmias et al. Neurosci Lett. 2008, 436:145; Bates et al. Neurosci Lett. 2009, 458:140; Corneveaux et al. Neurobiol. Aging 2008, Sep. 11, e-published).

In the next decades, the aging population of the industrialized countries will suffer from an increase of different forms of memory impairments like Alzheimer's disease, aging-related cognitive decline, mild cognitive impairment, or vascular dementia. Novel treatment options are desperately needed. KIBRA may well serve as a novel pharmacological target to treat multiple forms of memory impairment, as it is linked to Alzheimer's disease and might be accessible pharmacologically (WO 2007/120955).

The role of KIBRA for neuronal plasticity is further underlined by the observation that KIBRA interacts with protein kinase C zeta (PKC zeta), a molecule crucially involved in neuronal plasticity (Büther et al. Biochem Biophys Res Commun. 2004, 317:703). It was also shown that PKC zeta can phosphorylate KIBRA. To date, numerous scientific publications could demonstrate that PKC zeta, and also protein kinase M zeta (PKM zeta), the constitutively active form of PKC zeta, are both involved in learning and memory. A series of elegant papers fosters the influence of the kinase on memory maintenance in animals in the hippocampus (Drier et al. Nat Neurosci. 2002, 5:316; Pastalkova et al. Science 2006, 313:1141; Serrano et al. PLoS Biol. 2008, 6:2698) and in the cortex (Shema et al. Learn Mem. 2009, 16:122; Shema et al. Science 2007, 317:951). Moreover, specific inhibition of the PKM zeta activity disrupts the maintenance of LTP (Serrano et al. 3 Neurosci. 2005, 25:1979; WO 01/80875; WO 02/87417), a process regarded as a direct correlate of memory, whereas the activation of PKM zeta leads to increased LTP (Ling et al. Nat Neurosci. 2002, 5:295).

There is a need for reliable, efficient and non-laborious in vitro or cell based methods for the identification of new memory modulating compounds and means which can be performed in a medium to high throughput setup. These screening methods need easy to measure and robust readouts to allow multiple testing of compounds or treatments. The embodiments characterized in the claims and herein below provide such methods based on the linkage between KIBRA and memory performance.

Accordingly, the present invention relates to in vitro or cell based methods for the identification of compounds and means capable of modulating memory performance or cognition based on the assessment of changes in KIBRA expression.

The term “expression” refers to gene expression as well as protein expression. Gene expression can be assessed e.g. by RT-PCR or micro array technologies measuring the amount of individual gene transcripts. Protein expression can be measured e.g. within an antibody based ELISA assay quantifying the amount or concentration of individual proteins. The expression level assessed according to the present invention is influenced by the synthesis rate as well as by the degradation rate or the stability of the transcript and the protein.

The term “modulation” or “modulate” means the partial or essentially complete increase or decrease.

The terms “compound” and “substance” are to be understood broadly and means, in a general manner, all the material means and substances, which can be contacted to cultured cells, e.g. but without any claim of completeness: low molecular weight compounds, peptides, polypeptides, antibodies, nucleic acids, antisense nucleic acids, siRNA, aptameres, natural or artificial transcription factors, nucleic acid constructs, vectors, viral constructs, or liposomal means.

The inventors could demonstrate that KIBRA gene expression is causatively linked to memory performance. In animal studies, rats whose KIBRA expression was elevated in the hippocampal formation, an area central to memory related pathways, maintained memory significantly better than animals of the control group (Example 1). Detailed analysis of the data revealed that KIBRA over-expression impacts the memory storage, both relevant during acquisition and retrieval in different learning paradigms, such as the Sacktor disk and the Morris water maze experiment. Accordingly, KIBRA gene expression can be enhanced in situations of increased plasticity in the hippocampus, for example by application of growth factors like G-CSF (granulocyte-colony stimulating factor) (Example 2). Increased KIBRA levels have also been seen in experiments where rats received physical rehabilitation training after cerebral ischemia, implying that increased plasticity linked to increases in KIBRA levels is not only useful for cognition and memory, but plays also a role in CNS-inherent plastic reactions to injury and disease, such as ischemic disease. This signifies that KIBRA expression levels are a correlate of increased plasticity in the hippocampus and brain. These evidences clearly suggest that enhanced KIBRA levels are linked to improved cognitive performance and general CNS plasticity.

Additionally, the inventors found that over-expression of KIBRA in cells results in both increased expression and enhanced activity of PKC zeta in cell culture experiments (Example 3). Both, increased PKC zeta levels and enhanced activity can be directly correlated with improved memory performance and vice versa (Serrano et al. J. Neurosci. 2005, 25:1979; Ling et al. Nat Neurosci. 2002, 5:295; WO 01/80875; WO 02/87417).

Accordingly, one aspect of the invention is the provision of in vitro or cell culture based assays which allow to screen for KIBRA expression modulating compounds which are a highly attractive approach for the identification of memory modulators. In particular, compounds capable to enhance KIBRA expression or KIBRA activity are interesting candidates for improving cognitive performance in subjects.

In one aspect, the present invention provides methods for identification of memory and/or cognition modulating test compounds, which comprise
a) contacting said test compound with cultured cells and
b) measuring the modulation of KIBRA expression within the test cells.

To screen for compounds able to change KIBRA-expression levels one would preferably use a cultured cell line, preferentially cell lines that naturally express KIBRA, most preferred kidney or CNS derived cell lines (e.g. PC12 cells, SH-SY5Y cells, or HEK cells) or primary cells, preferentially cells that natively express KIBRA, most preferred neural cells, e.g. hippocampal cells, as the screen addresses neuronal processes, or kidney cells, which both are known to express KIBRA protein (Kremerskothen, J. 2003; Biochem Biophys Res Commun, 2003. 300(4))

KIBRA expression can be measured on the gene expression level (amount of mRNA) by methods known in the art such as RT-PCR (e.g. Roche LightCycler®), micro array technology (e.g. Affymetrix GeneChip®) or Northern-blotting methods, using probes complementary to the KIBRA cDNA sequence (e.g. human KIBRA (SEQ ID NO: 1), mouse KIBRA (SEQ ID NO: 3), or rat KIBRA (SEQ ID NO: 5); KIBRA cDNA sequences of further species can be identified by homology screening in cDNA libraries know to a skilled person). Such methods allow for quantitative evaluation of individual mRNA levels, and can be normalized to a reference or “housekeeping” target if necessary. Suitable primer pairs for LightCycler® based RT-PCR quantification of human KIBRA are e.g. 5′-CAC CAG AAG ACC TTA AGA GTC GA-3′ (SEQ ID NO: 7) and 5′-TCA CTA TCA CTC CGA TTC AGC C-3′ (SEQ ID NO: 8) and for mouse and rat KIBRA e.g. 5′-GAA GGA GCT GM GGA GCA TTT-3′ (SEQ ID NO: 9) and 5′-CCT GAA AGA CTG CAC TTC TGG-3′ (SEQ ID NO: 10). A skilled person can derive further primer pairs for these and other species. Advantageously, micro array technologies allow comparisons of KIBRA levels to many other genes, and thus conclusions as to the specificity of the effects caused by a test compound.

Alternatively, alteration of KIBRA gene expression can be assessed also by using cells transfected by a reporter gene construct, wherein the reporter gene is under the control of the KIBRA promoter (as described in Example 4). Suitable reporter genes are e.g. without any claim to completeness: luminescence causing enzymes (such as firefly luciferase, renilla luciferase, or bacterial luciferase operon (Photorhabdus luminescens IuxCDABE)), fluorescent proteins (such as green fluorescent protein (Aequorea victoria) and derivatives thereof (e.g. EYFP), or red fluorescent protein (Discosoma sp.)), substrate converting enzymes (such as beta-galactosidase, chloramphenicol acetyltransferase, beta-glucuronidase, or alkaline phosphatase), or resistance-conferring genes or else genes for growth selection.

As KIBRA promoter one can use the 3 kb gene fragment upstream the KIBRA start codon (e.g. human KIBRA upstream gene fragment (SEQ ID NO: 11), mouse KIBRA upstream gene fragment (SEQ ID NO: 12), or rat KIBRA upstream gene fragment (SEQ ID NO:13)) or any truncated fragment thereof with a length of at least 500, at least 300, or at least 100 bp, including homologue nucleic acids which are at least 90% identical thereto, whereas the truncated fragments are preferably adjacent to the KIBRA start codon or to the KIBRA transcription start. FIG. 1 shows an alignment of the human KIBRA upstream gene fragment (SEQ ID NO: 11), the mouse KIBRA upstream gene fragment (SEQ ID NO: 12), and the rat KIBRA upstream gene fragment (SEQ ID NO:13). The truncated fragments suitable for the use within the reporter construct according to the invention are preferably located in regions of comparable high homology of this alignment. Table 1 summarizes the localization of transcription factors binding sides predicted within the approximately 1 kb genomic upstream region of KIBRA for human, mouse and rat. These regions are particularly interesting for selection of truncated fragments suitable for the use within the reporter construct according to the invention. One skilled in the art can isolate KIBRA promoter gene fragments of further species by homology screening using KIBRA cDNA probes and species specific genomic libraries and subsequent cloning of the genomic fragments upstream the KIBRA coding region. KIBRA-promoter containing constructs can be stably transfected in the test cell lines or transiently transfected in the test cells. Preferably, the KIBRA promoter should be species-specific for the cells used in the assay. The specificity of the promoter sequence is important in order to bind the complex set of transcription factors in the cells. Such nucleic acid constructs comprising a functional reporter gene under the control of a KIBRA promoter fragment, corresponding vectors, and host cells containing such construct are also part of the present invention. The KIBRA promoter fragment can comprise the complete 3 kb gene fragment upstream the KIBRA start codon, or one or more of the above specified truncated fragments thereof.

As a further aspect of the present invention, one can also add genomic sequences 3′ to the KIBRA stop codon to such a reporter construct. Often, enhancer sites are located downstream of the coding sequences. Preferably, about 3 kb of the sequence 3′ to the KIBRA stop codon are added. In some embodiments, any truncated fragment thereof with a length of at least 2500, 2000, 1500, 1000, 500, 300, or 100 bp is added.

In addition to using the promoter, one can also assay other genomic sequences, e.g. intron 9 of the KIBRA gene. This intron will be cloned into the luciferase open reading frame, and render the luciferase ORF non-functional unless intron 9 is spliced from the pre-mRNA. As above, substance libraries can be assayed that result in enhanced luciferase activity. In the same way, all other introns in the KIBRA gene can be assayed.

TABLE 1
Positions of predicted transcription factor binding sides within the approximately
1 kb 5′ upstream genomic region of the human, mouse, and rat KIBRA gene.
The position numbering refers to the sequences SEQ ID NO: 11, SEQ ID NO: 12,
and SEQ ID NO: 13, respectively.

| Transcription | | | |
|---|---|---|---|
| Factor | Human | Mouse | Rat |
| NFKB1 | 2880-2890; 2847-2857; | 2849-2859; 2563-2573 | 2882-2892; 2849-2859; |
| | 2499-2509; 2303-2313 | | 2572-2582 |
| SPZ1 | 2904-2918; 2880-2894; | 2905-2919; 2861-2875; | 2905-2919; 2882-2896; |
| | 2859-2873; 2834-2848; | 2836-2850; 2793-2807; | 2849-2863; 2793-2807; |
| | 2795-2809; 2779-2793; | 2768-2782; 2706-2720; | 2768-2782; 2706-2720; |
| | 2679-2693; 2662-2676; | 2649-2678; 2625-2639; | 2689-2703; 2667-2681; |
| | 2633-2647; 2608-2622; | 2598-2612; 2577-2591; | 2607-2621; 2586-2600; |
| | 2585-2599; 2534-2548; | 2544-2558; 2512-2526; | 2553-2567; 2521-2535; |
| | 2513-2527; 2480-2494; | 2484-2498; 2463-2477; | 2493-2507; 2472-2486; |
| | 2448-2462; 2420-2434; | 2434-2448; 2412-2426; | 2420-2434; 2253-2267; |
| | 2399-2413; 2370-2384; | 2243-2257; 2216-2230; | 2227-2241; 2194-2208 |
| | 2334-2348; 2303-2317; | 2183-2197 | |
| | 2254-2268; 2221-2235; | | |
| | 2097-2111; 2033-2047; | | |
| | 1947-1961 | | |
| ZNF219 | 2885-2896; 2865-2876; | 2866-2877; 2461-2472; | 2886-2897; 2866-2877; |
| | 2397-2408; 2095-2106 | 2242-2253 | 2470-2481; 2251-2262 |
| MINI19_B | 2875-2895; 2833-2853; | 2908-2928; 2877-2897; | 2908-2928; 2877-2897; |
| | 2807-2827; 2712-2732; | 2835-2855; 2797-2817; | 2835-2855; 2803-2823; |
| | 2668-2688; 2633-2653; | 2730-2771; 2698-2718; | 2730-2771; 2694-2714; |
| | 2589-2609; 2249-2269; | 2667-2687; 2585-2605; | 2667-2687; 2594-2614; |
| | 2167-2187; 2028-2048 | 2429-2449; 2386-2406; | 2376-2396; 2323-2343 |
| | | 2314-2334 | |
| CBF2 | 2906-2921; 2870-2885; | 2907-2922; 2872-2887; | 2907-2922; 2870-2885; |
| | 2838-2853; 2818-2833; | 2837-2852; 2812-2827; | 2837-2852; 2812-2827; |
| | 2777-2808; 2743-2758; | 2795-2810; 2766-2781; | 2795-2810; 2766-2781; |
| | 2680-2695; 2653-2668; | 2739-2754; 2704-2719; | 2737-2752; 2704-2719; |
| | 2636-2651; 2616-2631; | 2687-2702; 2670-2685; | 2682-2697; 2646-2677; |
| | 2570-2585; 2532-2547; | 2650-2665; 2623-2638; | 2605-2620; 2587-2602; |
| | 2514-2529; 2478-2493; | 2596-2611; 2578-2593; | 2551-2566; 2534-2549; |
| | 2441-2456; 2421-2436; | 2542-2557; 2494-2509; | 2503-2518; 2463-2478; |
| | 2390-2405; 2368-2383; | 2454-2469; 2432-2447; | 2438-2453; 2376-2391; |
| | 2332-2347; 2288-2303; | 2367-2382; 2306-2321; | 2315-2330; 2153-2184; |
| | 2252-2267; 2222-2237; | 2090-2105 | 2100-2115 |
| | 2188-2203; 2159-2174; | | |
| | 2038-2053; 2021-2036; | | |
| | 1960-1975 | | |
| WT1Q6 | 2911-2919; 2896-2904; | 2905-2913; 2886-2894; | 2905-2913; 2878-2895; |
| | 2876-2893; 2866-2874; | 2867-2875; 2855-2863; | 2867-2875; 2855-2863; |
| | 2852-2860; 2834-2842; | 2836-2844; 2805-2813; | 2836-2844; 2805-2813; |
| | 2809-2817; 2795-2803; | 2793-2801; 2768-2776; | 2793-2801; 2768-2776; |
| | 2779-2787; 2740-2748; | 2726-2734; 2706-2714; | 2726-2734; 2706-2714; |
| | 2728-2736; 2691-2699; | 2696-2704; 2675-2683; | 2687-2704; 2667-2675; |
| | 2679-2687; 2668-2676; | 2649-2666; 2616-2633; | 2637-2645; 2627-2635; |
| | 2655-2663; 2645-2653; | 2594-2602; 2583-2591; | 2603-2611; 2592-2600; |
| | 2633-2641; 2618-2626; | 2560-2568; 2550-2558; | 2569-2577; 2559-2567; |
| | 2608-2616; 2596-2604; | 2539-2547; 2510-2518; | 2548-2556; 2538-2546; |
| | 2585-2593; 2572-2580; | 2490-2498; 2463-2471; | 2519-2527; 2499-2507; |
| | 2558-2566; 2534-2542; | 2432-2449; 2422-2430; | 2472-2480; 2441-2458; |
| | 2519-2527; 2496-2504; | 2398-2406; 2378-2386; | 2430-2438; 2406-2414; |
| | 2480-2488; 2468-2476; | 2366-2374; 2351-2359; | 2387-2395; 2375-2383; |
| | 2446-2454; 2426-2434; | 2325-2333; 2281-2289; | 2360-2368; 2334-2342; |
| | 2399-2407; 2370-2378; | 2243-2251; 2230-2238; | 2290-2298; 2254-2262; |
| | 2357-2365; 2334-2351; | 2216-2224; 2201-2209; | 2241-2249; 2227-2235; |
| | 2308-2316; 2268-2276; | 2183-2191; 2091-2099 | 2212-2220; 2194-2202; |
| | 2254-2262; 2227-2235; | | 2157-2165; 2101-2109 |
| | 2205-2213; 2190-2198; | | |
| | 2178-2186; 2097-2105; | | |
| | 2085-2093; 2075-2083; | | |
| | 2059-2067; 2044-2052; | | |
| | 2033-2041; 2023-2031; | | |
| | 2001-2009; 1978-1986; | | |
| | 1953-1961; 1938-1946 | | |
| PAX5 | 2825-2852; 2755-2782; | 2813-2840; 2773-2800; | 2813-2840; 2773-2800; |
| | 2561-2588 | 2488-2515 | 2658-2685; 2628-2655; |
| | | | 2497-2524; 2429-2456 |
| AP2_Q6 | 2911-2922; 2885-2896; | 2920-2931; 2873-2884; | 2920-2931; 2887-2898; |
| | 2871-2882; 2843-2854; | 2845-2856; 2816-2827; | 2873-2884; 2856-2867; |
| | 2814-2825; 2800-2811; | 2802-2813; 2767-2778; | 2816-2827; 2802-2813; |
| | 2781-2792; 2730-2741; | 2746-2757; 2692-2703; | 2767-2778; 2746-2757; |
| | 2716-2727; 2667-2678; | 2644-2655; 2613-2636; | 2728-2739; 2684-2695; |
| | 2598-2621; 2556-2567; | 2556-2567; 2529-2540; | 2622-2645; 2565-2576; |
| | 2492-2503; 2465-2476; | 2488-2511; 2461-2472; | 2538-2549; 2497-2520; |
| | 2448-2459; 2428-2439; | 2444-2455; 2419-2430; | 2470-2481; 2453-2464; |
| | 2397-2408; 2380-2391; | 2387-2398; 2363-2374; | 2427-2438; 2372-2383; |
| | 2349-2360; 2329-2340; | 2239-2250; 2198-2209; | 2251-2262; 2209-2220; |
| | 2308-2319; 2249-2260; | 2093-2104 | 2188-2199; 2158-2169; |
| | 2218-2229; 2039-2050; | | 2103-2114 |
| | 1995-2006; 1955-1966; | | |
| | 1940-1951 | | |
| MA0079 | 2883-2892; 2864-2873; | 2909-2918; 2898-2907; | 2909-2918; 2898-2907; |
| | 2844-2853; 2820-2829; | 2846-2855; 2822-2831; | 2885-2894; 2848-2857; |
| | 2796-2805; 2776-2785; | 2798-2817; 2782-2791; | 2822-2831; 2798-2817; |
| | 2764-2773; 2743-2752; | 2769-2778; 2756-2765; | 2782-2791; 2769-2778; |
| | 2713-2722; 2689-2708; | 2735-2744; 2693-2722; | 2756-2765; 2731-2740; |
| | 2669-2678; 2653-2662; | 2662-2671; 2650-2659; | 2703-2722; 2689-2698; |
| | 2634-2643; 2615-2624; | 2626-2635; 2581-2590; | 2668-2677; 2646-2655; |
| | 2559-2568; 2523-2532; | 2563-2572; 2545-2554; | 2628-2637; 2590-2599; |
| | 2499-2508; 2481-2490; | 2513-2522; 2489-2498; | 2572-2581; 2554-2563; |
| | 2448-2457; 2425-2444; | 2467-2476; 2408-2417; | 2522-2531; 2498-2507; |
| | 2403-2412; 2381-2390; | 2351-2360; 2243-2252; | 2476-2485; 2433-2442; |
| | 2354-2363; 2335-2344; | 2184-2193; 2090-2099 | 2360-2369; 2190-2199; |
| | 2304-2313; 2255-2264; | | 2169-2178; 2100-2109 |
| | 2205-2214; 2059-2068; | | |
| | 2041-2050; 2024-2033 | | |
| KROX6 | 2883-2896; 2862-2875; | 2864-2877; 2792-2805; | 2885-2898; 2864-2877; |
| | 2781-2794; 2667-2680; | 2767-2780; 2644-2657; | 2792-2805; 2767-2780; |
| | 2640-2653; 2610-2623; | 2490-2503; 2461-2474; | 2683-2696; 2499-2512; |
| | 2426-2439; 2397-2410; | 2242-2255; 2091-2104 | 2470-2483; 2251-2264; |
| | 2306-2319; 2095-2108; | | 2193-2206; 2101-2114 |
| | 2039-2052 | | |
| CACD | 2884-2891; 2839-2846; | 2885-2892; 2853-2860; | 2886-2893; 2853-2860; |
| | 2797-2804; 2788-2795; | 2841-2848; 2799-2806; | 2799-2806; 2762-2769; |
| | 2727-2734; 2647-2654; | 2762-2769; 2651-2658; | 2725-2732; 2641-2648; |
| | 2610-2617; 2595-2602; | 2620-2627; 2549-2556; | 2629-2636; 2558-2565; |
| | 2518-2525; 2485-2492; | 2514-2521; 2489-2496; | 2523-2530; 2498-2505; |
| | 2450-2457; 2425-2432; | 2468-2475; 2353-2360; | 2477-2484; 2362-2369; |
| | 2404-2411; 2307-2314; | 2247-2254 | 2256-2263; 2196-2203 |
| | 2256-2263; 2207-2214; | | |
| | 2100-2107; 2061-2068; | | |
| | 2035-2042 | | |
| LRF2 | 2883-2891; 2862-2870; | 2917-2925; 2885-2893; | 2917-2925; 2885-2893; |
| | 2850-2858; 2839-2847; | 2864-2872; 2852-2860; | 2864-2872; 2852-2860; |
| | 2812-2820; 2499-2507; | 2841-2849; 2814-2822; | 2814-2822; 2690-2707; |
| | 2453-2470; 2401-2409; | 2698-2706; 2631-2639; | 2640-2648; 2628-2636; |
| | 2359-2367; 2347-2355; | 2619-2627; 2563-2571; | 2572-2580; 2535-2543; |
| | 2334-2342; 2307-2315; | 2513-2521; 2465-2473; | 2522-2530; 2474-2482; |
| | 2224-2232; 2101-2109; | 2435-2443; 2425-2433; | 2443-2451; 2433-2441; |
| | 2029-2037; 2001-2009 | 2369-2377; 2248-2256; | 2378-2386; 2257-2265; |
| | | 2204-2212 | 2215-2223; 2194-2202; |
| | | | 2154-2162 |

Recently, it has become obvious that the level of gene expression regulation on the mRNA level is much higher and more complex than previously assumed. mRNA stability can be affected by proteins binding to the 3′UTR for example. Therefore, the present invention also provides methods for identification of memory and/or cognition enhancing test compounds, which comprise the constitutive expression of full length KIBRA mRNA in the culture cells, contacting said cells with the test compound, and measuring the amount of KIBRA transcript in comparison to control cell (Example 5). Suitable expression constructs comprise the full length KIBRA cDNA (e.g. human KIBRA (SEQ ID NO: 1), mouse KIBRA (SEQ ID NO: 3), or rat KIBRA (SEQ ID NO: 5) under the control of a eukaryotic promoter such as CMV or SV40. Said KIBRA cDNA comprises the coding region and at least 50 bp of the corresponding 5′UTR and/or at least 100 bp of the corresponding 3′UTR. The amount of the KIBRA transcript can be measured e.g. with RT-PCR based methods such as LightCycler®. In order to examine this stability effect isolated from potential influences on the KIBRA promoter, one can use for this assay cell without exogenous KIBRA expression, or cells derived from a species whose KIBRA sequences distinguishes sufficiently enough from the one use in the expression construct, and thereby, allow for separate detection.

Alternatively, alteration of KIBRA expression can be assessed on the protein level (amount of protein) by methods known in the art such as ELISA, Western-blot, or immunoprecipitation using specific anti-KIBRA antibody. Detection of KIBRA protein level can be performed by using KIBRA specific antibodies. Such antibodies can be generated form mouse hybridoma cells, which have been generated e.g. by the immunization of mice using KIBRA peptides, in order to receive specific monoclonal antibodies. Alternatively, polyclonal antibodies isolated from the serum of animals immunized with KIBRA protein or peptides thereof can be used accordingly. Suitable KIBRA peptides can be derived form the KIBRA protein sequences (e.g. human KIBRA protein (SEQ ID NO: 2), mouse KIBRA protein (SEQ ID NO: 4), or rat KIBRA protein (SEQ ID NO: 6)).

Furthermore, the present invention also provides methods for identification of memory and/or cognition enhancing test compounds, which comprise the constitutive expression of KIBRA protein or a KIBRA fusion protein in the cultured cells, contacting said cells with the test compound, and measuring the amount of KIBRA protein or KIBRA fusion protein in comparison to control cell (Example 6). Compounds that influence the stability of KIBRA protein can be identified within this assay method. The amount of KIBRA protein and KIBRA fusion protein can be quantified by antibody based methods such as ELISA using specific anti-KIBRA antibodies. In KIBRA fusion proteins the KIBRA protein is linked to a tag peptide such as myc-tag or FLAG-tag, which allows detection with commercially available antibodies, or to a reporter protein such as luciferase which allows direct quantification of the fusion protein.

Also, KIBRA expression can be monitored utilizing transgenic cells whereas the KIBRA gene is partly replaced by a reporter construct, such as the luciferase cDNA resulting in a chimaeric protein under the control of the original KIBRA promoter of the cells. One such construct could be a fusion of the full-length luciferase cDNA subsequently to the 3′-end of the KIBRA coding region.

A test compound is considered as screening hit according to the invention if KIBRA expression is modulated in the cultured cells by at least 20%, compared to control cells and/or if KIBRA expression is modulated statistically significantly in cultured cells compared to control cells. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 95%. The p-values are, preferably, 0.05.

Test compounds for this assay method can be for example polypeptides, proteins (including chimeric proteins), antibodies, nucleic acids (e.g. siRNA), gene therapy approaches (virus) or small molecules. Preferable for a high throughput appoach are small molecules, with a molecular weight of less than 2,000 g. Compounds are generally diluted in salt buffer of physiological concentration (e.g. PBS), hydrophobic compounds, like fat-soluble vitamins, can be diluted in suitable solvents, e.g. 20% Solutol HS (BASF, Ludwigshafen, Germany), for efficient application to the cell culture.

In the view of the above, a method according to the invention that identifies compounds enhancing KIBRA expression or activity is a promising approach for treating and/or preventing cognition impairments, including non-pathological forms like “age related memory loss” (ARML). Cognitively impaired people have difficulty with one or more of the basic functions of their brain, such as perception, memory, concentration and reasoning skills. Common causes of cognitive impairment include Alzheimer's disease and related dementias, stroke, Parkinson's disease, brain injury, brain tumor or HIV-associated dementia.

Accordingly, one aspect of the present invention is the use of compounds, identified by a method of the invention as being capable to enhance the KIBRA expression, for the manufacturing of a pharmaceutical preparation for enhancing memory and cognitive ability, and thereby, treating disease such as Alzheimer's disease, dementia, or “mild cognitive impairment” (MCI) and also non-pathological ARML.

Also, one aspect of the present invention is a method for improving memory or cognitive ability in a subject, the method comprising administering to said subject in need thereof, a therapeutically effective amount of a compound, identified by a method of the invention as being capable to enhance the KIBRA expression, particularly, whereas said subject suffer from Alzheimer's disease, dementia, MIC, or non-pathological ARML.

### BRIEF DESCRIPTION OF THE FIGURES

FIG. 1: Alignment of the approximately 1 kb genomic sequence upstream of the coding region start of the human, mouse and rat KIBRA promoter region (KIBRA_hs: human genomic sequence, bases 1940 to 3000 of SEQ ID NO: 11; KIBRA_mm: mouse genomic sequence, bases 2093 to 3000 of SEQ ID NO: 12; KIBRA_rn: rat genomic sequence, bases 2103 to 3000 of SEQ ID NO: 13). Asterisks mark positions of the alignment with bases identical over all three species.

FIG. 2: Sacktor disk results for KIBRA over-expressing (KIBRA) and control (CONTROL) animal; bars show the time to enter the shock sector in seconds for the rats of the two groups for the acquisition (9^{th} trial) and for the recall trial after 24 h. KIBRA over-expressing animals perform significantly better in the recall trial by avoiding the shock sector for a longer time. Error bars represent the SEM.

FIG. 3 A: Depiction of traces of the test rat obtained during water maze learning, at the beginning of the learning trials and at the end. Whereas in the initial trial, the rat searches for the hidden platform throughout the complete basis, at the end of the trials, the rat knows where to find the hidden platform.

FIG. 3 B: Comparison of learning speed (mean of the time (sec) to platform for the test rats) in the first 4 learning runs between KIBRA over-expressing rats (left panel) and control rats (right panel). Error bars represent the SEM. After initial learning trials, KIBRA over-expressors need less time to find the hidden platform compared to the control animals.

FIG. 4: Induction of PKC zeta activity by KIBRA. The hatched bars show the PKC zeta kinase activity in the absence (PKCzeta) and in the presence (PKCzeta+KIBRA) of KIBRA over-expression, whereas the PKC zeta activity was measured, after affinity purification and normalization to the PKC zeta protein amount, in various dilutions (1:10, 1:25 and 1:50). The black bar (buffer) is the base line control of the PKC zeta kinase assay without addition of any PKC zeta protein. The white bar (PKCzeta) shows the positive control of the PKC zeta kinase assay in the presence of 5 ng PKC zeta protein.

FIG. 5: Result of the luciferase assay after stimulation of SHSY5Y cells transfected with the KIBRA promoter construct with the compounds piperine and retinoic acid (1 μM each, 48 h). Piperine (black box), but not retinoic acid (white box), is able to induce an elevation of the luciferase signal. Control (hatched box): untreated cells.

FIG. 6: Rat neuronal stem cells were stimulated with piperine and retinoic acid for 48 h. Quantitative PCR of these cells shows, that the amount of native KIBRA mRNA is elevated by piperine (black box), but not by retinoic acid (white box). Untreated cells (hatched box) served as control.

FIG. 7: YAP elevates the luciferase reporter signal in SHSY5Y cells. SHSY5Y cells transfected with a reporter construct (KIBRA-5′ luciferase KIBRA-3′) according to the invention; co-transfected with an expression vector containing the Yap ORF (hatched box) and co-transfected with an empty expression vector as control (white box).

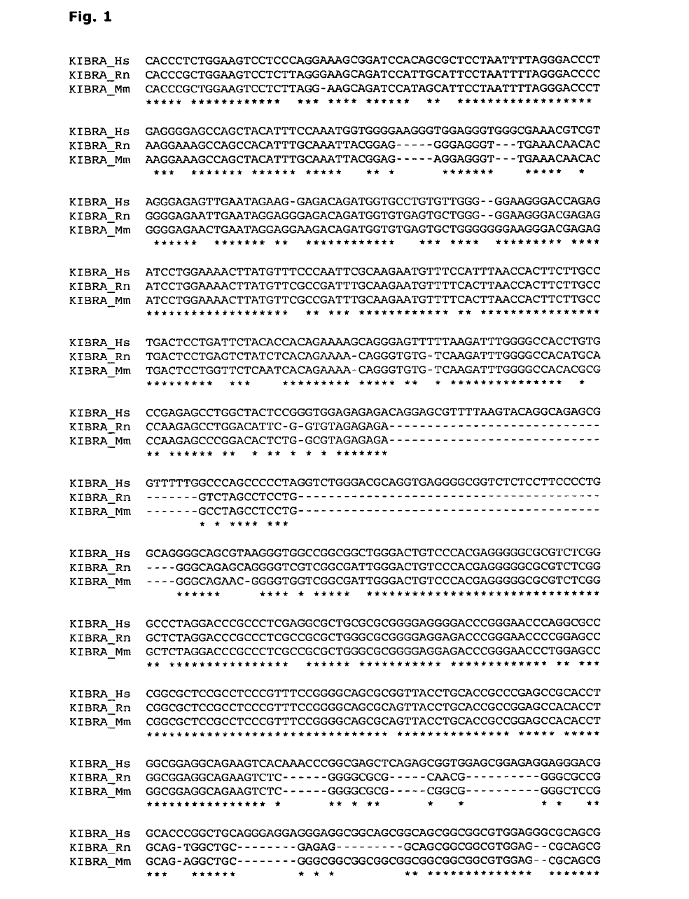

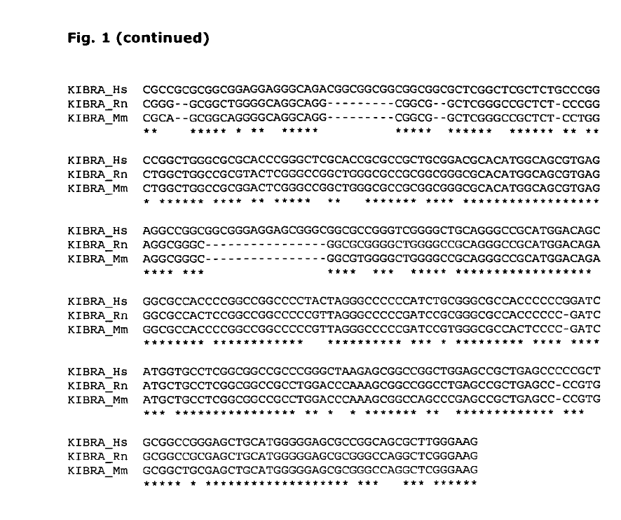

### EXAMPLES

### Example 1

### Over-Expression of KIBRA in the Hippocampus of Rats

KIBRA was over-expressed in the hippocampal region of rats. The over-expression was mediated by Adeno-associated virus 2 (AAV2) KIBRA injection via stereotactic surgery in the hippocampal region of both brain hemispheres of adult male rats (3 month of age). After three weeks animals either treated with AAV KIBRA or an AAV expressing GFP (n=20, each group), were subjected to behavioural tests.

KIBRA over-expressors showed a strong improvement in memory performance in a specific form of active place avoidance, herein called “Sacktor disk”. The Sacktor disk provides the experimental advantages of rapid hippocampus-dependent acquisition and persistent hippocampus-dependent recall (Pastalkova et al. Science 2006, 313:1141). The apparatus consists of a slowly rotating platform disk (1 rpm), open to the room environment. The rotation of the platform carries the animals into a 60° shock sector, which can be energized, forcing the animals to actively avoid the shock sector. A video camera connected with a tracking system is installed above the maze to record the movement of the animals.

For the analysis animals are first subjected to a habituation trial, in which the animals are exposed to the apparatus for 10 min without shock. After the habituation animals continue with successive training trials, in which the animals receive an electric shock every time the animals run into the shock zone (0.6 mA for 0.5 s every 1.5 s). Training consists of eight 10 min training trials, separated by 10 min rest intervals in their home cage.

Retention of long term memory is tested after 24 h in a single probe trial for 10 min, in the absence of shock. The performance of the animals is determined by the time the animal first entered the shock zone, total visits of the shock zone and number of shocks (time in shock zone).

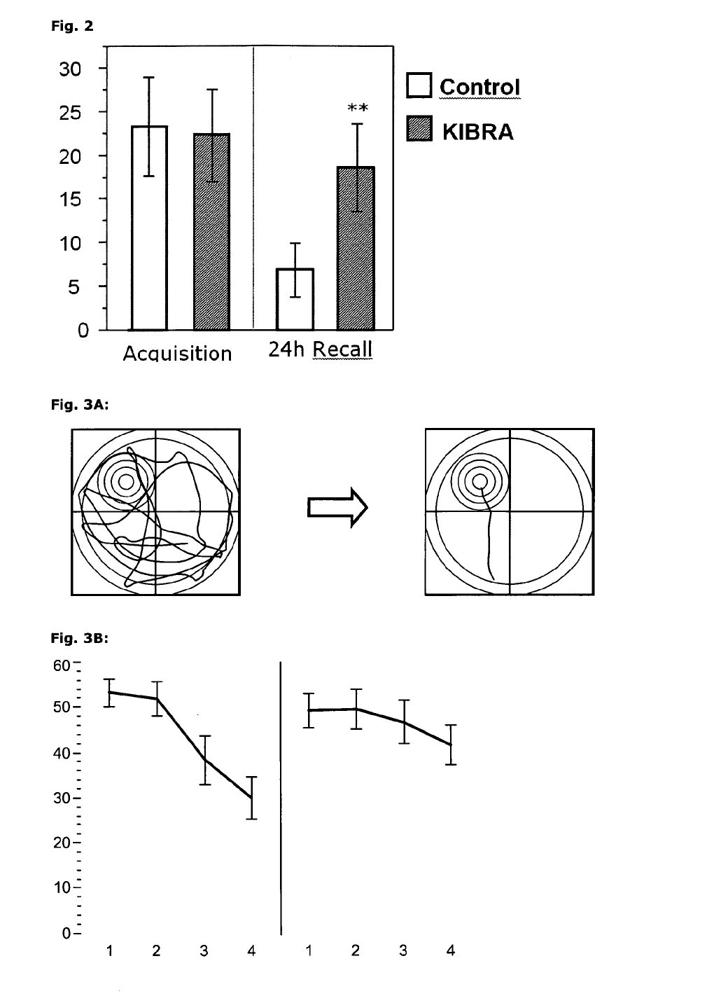

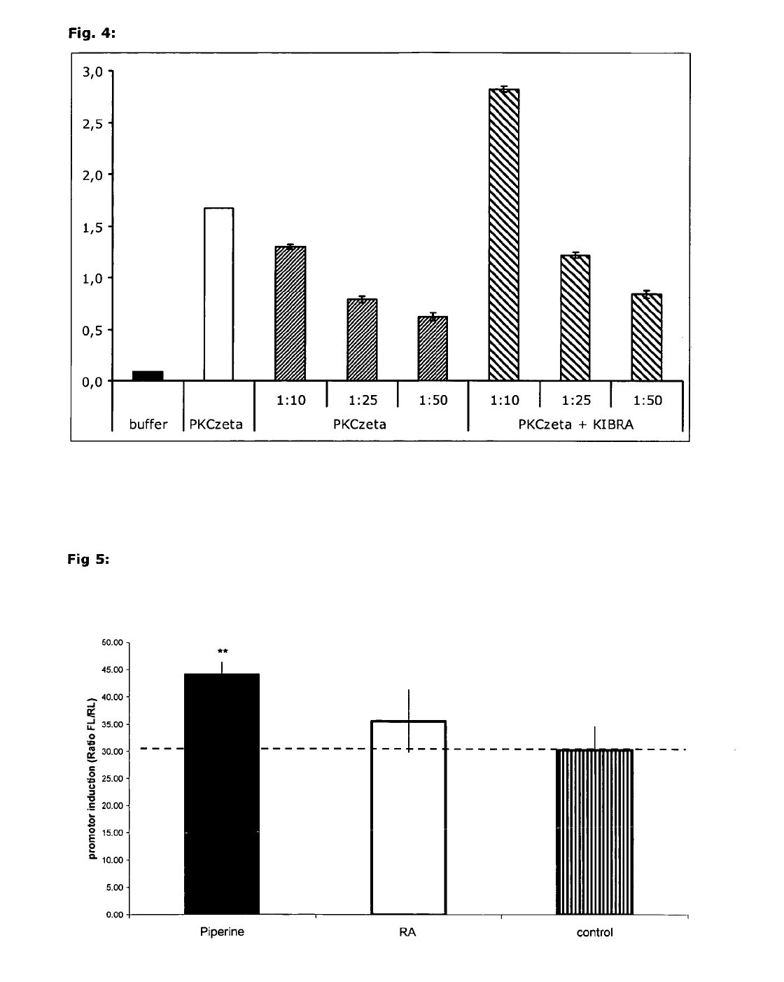

KIBRA AAV treated rats performed significantly better than the control group, as the animals avoided the shock zone for a longer time (FIG. 2). In line with the time KIBRA over-expressing animals to avoid the shock zone, KIBRA over-expressors also showed significantly less visits in the shock zone.

Another way to measure cognition and memory is the Morris water maze test. The Morris water maze has been extensively used to analyze spatial learning and memory (Devan et al. Neurobiol Learn Mem. 1996, 66:305). The experimental procedure involves training rodents to find a hidden platform in a large, circular pool of water.

A water maze (diameter 180 cm) was filled with an opaque mixture of black, water-soluble cola-paint and was heated to 25° C. for trials. A collapsible Plexiglas platform is used (12 cm×12 cm surface) extended from the floor of the tank to 1.5 cm below the surface of the water. The tank is exposed to the room environment with different visual cues are positioned at the north, east, south and west of the tank, respectively. A video camera connected with a tracking system is installed above the maze to record the movement of the animals.

In the training trials, the platform is placed at a fixed position, lowered to 1.5 cm below the surface of the water, that the animals were unable to see the platform. On each trial, the rat was placed in the pool along the perimeter of the tank facing the tank wall. The four entry points, North, East, South, and West, were randomized across trials, but the same sequence of start points was used for each rat. The animals were allowed to reach the platform in 60 sec on each trial. Trials ended when the rat located the escape platform, or when 60 sec had elapsed, at which time the rat was guided to the platform. In either case, the rat was allowed to remain on the platform for 30 sec before being returned to a holding cage for a 30-sec inter-trial interval. Animals are tested for 4 trials each day for 4 d.

The time animals needed to reach the platform during each training trial was measured. After the training animals underwent a probe trial on the 5^{th} day in which the platform was completely removed. The animals are placed to the opposite quadrant of the platform quadrant and allowed to search for the platform for 120 sec. The time the animals spent in the quadrant and the distance over time to the place where the platform was originally located was recorded, as well as the distance animals covert in the maze. Analysis of the experiment showed that animals injected with the AAV-Virus leading to KIBRA over-expression performed significantly better in learning the location of the hidden platform than animals injected with the control AAV-Virus. Using multiple linear regression analysis with the outcome variable TIME TO PLATFORM and with the factors VIRUS TREATMENT, TRIAL NUMBER, STARTING POSITION, and VIRUS TREATMENT*TRIAL NUMBER both VIRUS TREATMENT itself and the interaction term were significantly different (VIRUS TREATMENT: p=0.0045; VIRUS TREATMENT*TRIAL NUMBER: p=0.0152). FIG. 3 A shows an example of traces obtained during water maze learning, FIG. 3 B shows a comparison of learning speed in the first 4 learning runs, with a clear advantage for KIBRA over-expressors.

Thus, in two different experimental learning paradigms KIBRA over-expression resulted in a significant improvement in learning and memory performance.

### Example 2

### G-CSF Enhances KIBRA Expression

The neuroprotective and neuroregenerative molecule Granulocyte-Colony Stimulating Factor (G-CSF) has effects on cognition. Synergetic effects of granulocyte-colony stimulating factor and cognitive training on spatial learning and survival of newborn hippocampal neurons have also been shown (Diederich et al. PLoS One 2009, 4:5303). The role of G-CSF in the healthy brain has been examined by the characterization of G-CSF-deficient mice (Diederich et al. J Neurosci 2009, 29:11572). In the present example, we tested the pro-cognitive G-CSF in anxiogenic and learning and memory paradigms in the mouse. After AAV-mediated delivery of G-CSF into the hippocampus of mice, no anxiogenic effects were detected, whereas spatial reference and working memory was improved in the G-CSG-treated group.

AAV constructs expressing the human G-CSF protein were stereotactically injected bilaterally into the hippocampus of 10 C57BL\6 mice at four sites (AP−1.3 mm/L±1.8 mm/DV−2 mm and AP−0.7 mm/L±2.4 mm/DV−2 mm). For controls, 10 mice were injected with the empty vector.

In the T-maze, the non-matching-to-place paradigm (NMTP) was tested for spatial working memory. In the T-shaped plastic apparatus, mice were trained in 10 trials per day, consisting of two runs each. In the first run, mice were allowed to enter only one arm of the T-maze containing sweetened milk. In the second run, both arms were available for choice, with the food reward placed in the opposite arm. In the paddling pool for testing spatial reference memory, mice were placed in the circular apparatus of 115 cm in diameter with 12 exit holes, of which 11 holes were blocked. In 5 trials per day, the animals were trained to find the unblocked exit always located at 12 o'clock. After 7 days, the exit position was rotated to 4 o'clock. The time until the new exit position was found and the number of errors was recorded.

Analysis of the results showed that G-CSF-over-expressing animals perform superior to AAV-control-injected animals in the T-maze test described above.

To analyze what molecular mechanisms contributed to enhanced cognition induced by G-CSF, we conducted a gene expression study of the hippocampus tissue. Gene expression was analyzed after Laser-microdissection of the hippocampus, amplification of mRNA, and hybridization to micro arrays (GeneChip®, Affymetrix, Santa Clara, Calif., USA). Data were analyzed using Genesifter (Geospiza Inc., Seattle, Wash., USA). The KIBRA mRNA was found significantly induced by a factor of 3.09-fold (Affymetrix ID 1427261_at; HECT C2 and WW domain containing E3 ubiquitin protein ligase 1).

This example demonstrates that KIBRA can be induced by pharmacological means, and that an increase in KIBRA correlates to improved cognitive capabilities.

### Example 3

### PKC Zeta Activity is Regulated by KIBRA

To investigate the impact of KIBRA on PKC zeta activity itself, PKC zeta activity were determined in an enzyme assay. The kinase activity in the assay is detected by the phosphorylation of a substrate peptide by an antibody reaction according to manufactures instruction (HTScan PKC zeta kinase assay kit, Cell Signaling Technology Inc., Danvers, Mass., USA). The antibody is detected by a lanthanide-labeled secondary antibody, which can be measured in a fluorescent plate reader.

In order to analyze PKC zeta activity with and without KIBRA, COS cells over-expressing PKC zeta alone or in combination with KIBRA were lysed and PKC zeta was pulled down with respective PKC zeta binding beads. To normalize for equal pull down of PKC zeta, aliquots of the pull downs were subjected to western blot analysis and the resulting PKC zeta signal in the blot were used to normalize for comparable amounts in the activity assay.

Remarkably, the amount of PKC zeta that was co-immunoprecipitated was similar between cells only over-expressing PKC zeta to cells over-expressing PKC zeta and KIBRA.

Previous studies have shown that PKC zeta co-immunoprecipitates with KIBRA, and immunoreactive bands for KIBRA in the western blot analysis confirmed that PKC zeta pulldowns include KIBRA protein from cells also over-expressing KIBRA. When pulldowns from cells over-expressing PKC zeta were compared to cells over-expressing both PKC zeta and KIBRA for the phosphorylation of substrate peptide in the activity assay, a strong increase of PKC zeta activity by 50% was observed. Verification of specificity of the assay was determined by pulldown of a truncated inactive form of PKC zeta. Analysis of the kinase activity of the truncated form confirmed that the phosphorylation of the peptide was only mediated by PKC zeta and no other kinase attached to the beads.

Analysis of different amounts of pulldowns form cells with and without KIBRA finally confirmed a significant and strong increase of PKC zeta activity when KIBRA is present.

### Example 4

### In Vitro Analysis of KIBRA Expression in the Presence of a Test Compound Using Genomic Sequences

One approach to alter expression of a particular gene is to target regulatory sequences directly, or indirectly (e.g. via transcription factors, splicing factors, or enhancer factors). For this, a reporter gene (such as luciferase) can be cloned after the human KIBRA promoter (e.g. the first 3000 bp 5′ to the start codon). This construct will be transfected into cells, ideally SHSY-5Y cells. These cells will be grown in 96- or 384-well plates at densities of 5000-30000 cells. Substances will be applied to the cells at concentrations of 1 or 10 μM, in several replicates per substance. Luciferase amount present per well can be determined by luciferase plate readers. Substances that result in statistically significant elevation of luciferase activity beyond 20% are considered as “hits”. As a positive control, co-transfection of the transcription factor Yap into one set of samples can be performed. Co-transfection of Yap significantly elevates luciferase expression in our test system (FIG. 7).

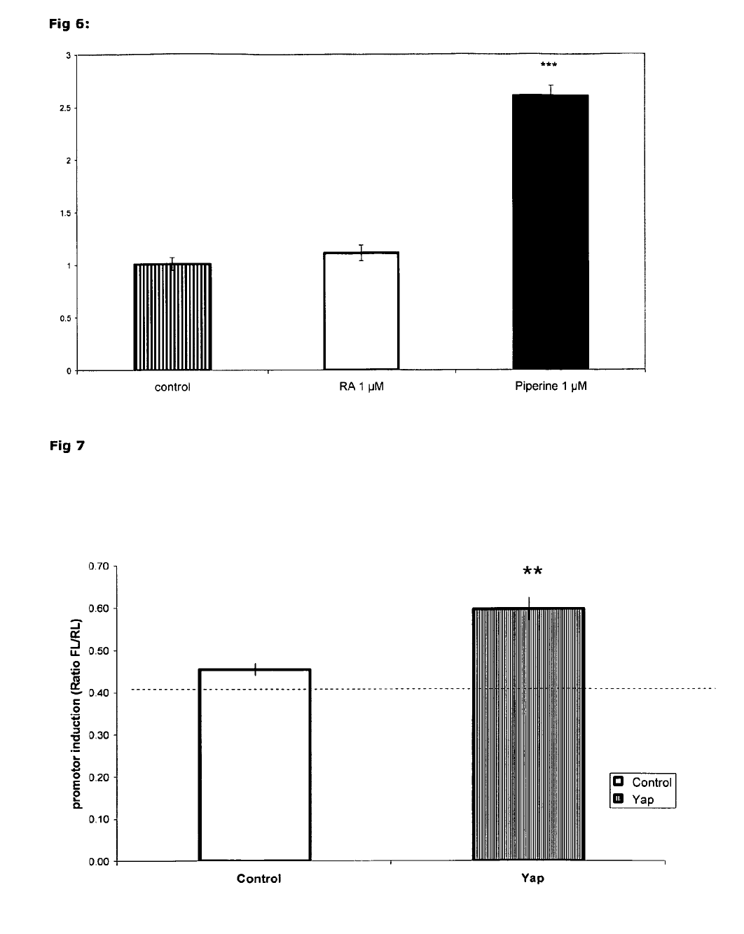

The test system has been verified in a set of experiments testing random compounds for induction of the luciferase reporter. For instance, piperine induced the construct significantly (FIG. 5). To verify that this induction faithfully replicates the behavior of the endogenous KIBRA promoter, cells stimulated with piperine were tested for induction of KIBRA mRNA by qPCR (FIG. 6). Correspondingly, retinoic acid, a known marker for neurogenesis failed to induce the KIBRA promoter construct.

### Example 5

### In Vitro Analysis of KIBRA Expression in the Presence of a Test Compound Using 5′ and 3′ mRNA UTR Sequences

For a screening approach, the full mRNA of human KIBRA will be cloned in a CMV vector, purified, and transfected into SHSY-5Y cells. Ideally, a stable transfectant will be established. Cells will then be grown on a 96 well plate at a density of 10.000-20.000 cells per well. Alternatively, cells can be grown on 384-well plates at a density of 5.000-10.000 cells per well. Small-molecule substances will be given to the wells in a concentration of 1 and 10 μM in 8 replicates per concentration. After 4 days in culture, RNA will be extracted from the cells, and analyzed by quantitative RT-PCR in comparison to an endogenous mRNA (cyclophilin). After statistical analysis, substances will be identified that produced a significant elevation (or depression) of KIBRA (WWC1) mRNA levels. A meaningful factor for up- or down-regulation is 20% and above, at a p value of <0.05. These substances (“hits”) will be further analyzed by doing a careful concentration curve to determine EC50, and will be checked for unspecific or unwanted effects such as cellular toxicity, apoptosis, downregulation or induction of other genes. The last point will be ensured by subjecting total mRNA extracted from cells harbouring the reporter plasmid (or having stably integrated the reporter plasmid into their genomes) after exposure to said substance to analysis by DNA-arrays, such as provided by the company Affymetrix. Statistical analysis of hybridization signals will then allow to determine relative specificity of the hit in question.

In an alternative screening approach, a reporter construct can be supplied that replaces the KIBRA open reading frame (ORF) with the coding sequence for a reporter protein such as luciferase, GFP, secreted luciferase, beta-galactosidase, or others. This construct can be transfected as described above, and the screening method conducted as described with the exception that the read-out will not be based on quantitative PCR, but on a reporter assay. Most favorably, this is a luciferase-based assay using plate readers. Results are treated as above, and substances further examined for specificity and toxicity. This approach will only identify substances that target the 5′ or 3′ UTR, while the first approach also allows to identify substances that target the whole mRNA sequence.

In a further alternative screening approach one relies on the endogenous gene expression. Such an approach can be performed exactly as described above but without transfecting the SHSY-5Y cells. Alternative to using SHSY-5Y cells, primary neurons can be employed, NG108 cells, NSC34 cells, PC12 cells, and other neural cells and cell lines known to the expert.

In these screening approaches, small molecule substances can be replaced by siRNA libraries, by peptide libraries, or by gene therapeutic viral constructs.

### Example 6

### In Vitro Analysis of KIBRA Expression in the Presence of a Test Compound Targeting the Stability of the KIBRA Protein

For a screening approach identifying compounds which influence the stability of the KIBRA protein, constructs can be used that either express KIBRA, or a fusion protein harbouring the full KIBRA sequence, and a reporter protein, such as luciferase. If only the KIBRA protein is used, the assay will be an ELISA. SHSY-5Y cells will be transfected with a plasmid harbouring an expression cassette for KIBRA. Cells will then be plated onto 96- or 384-well plates. Substances at concentrations of 1 μM or 10 μM will be applied to the cells in replicates. An ELISA using an antibody against human KIBRA will be performed. Read-outs will be statistically analyzed, and hits identified. Alternatively, the fusion part of the protein may be detected. If this is a small peptide tag, such as the FLAG, or myc epitope, an ELISA can be utilized detecting that unique tag.

If a reporter such as luciferase is to be detected, a luciferase assay using a plate reader will be employed.

The invention further relates to the following, non-limiting embodiments:
1. A method for identifying memory and/or cognition modulating substances, which comprises
   a. contacting said substance with cultured cells and
   b. assessing the modulation of KIBRA expression within the cells.
2. The method of 1, wherein the modulation of KIBRA expression on transcript level is assessed.
3. The method of 1, wherein the modulation of KIBRA expression on protein level is assessed.
4. The method of 2, wherein the endogenous KIBRA expression of the cells is quantified, by specific nucleic acid quantification methods, such as quantitative RT-PCR methods, by micro array methods, or by Northern blot methods.
5. The method of 2, wherein the cultured cells are transfected with an expression construct previous to the contacting of the cells with the test substance, whereas the expression construct comprises a reporter gene under the control of a KIBRA promoter.
6. The method of 5, wherein the KIBRA promoter is the 3 kb genomic region upstream of the KIBRA coding sequence or any truncated fragment with a length of at least 100 bp or a combination of two or more of these truncated fragments.
7. A expression vector comprising a reporter gene under the control of a KIBRA promoter.
8. The expression vector of 7, wherein the KIBRA promoter is the 3 kb genomic region upstream of the KIBRA coding sequence, or any truncated fragment with a length of at least 100 bp, or homologous thereof having at least 90% identity to said truncated fragments, or a combination of two or more of these truncated fragments.
9. A host cell containing the expression vector of 7 or 8.
10. The method of 2, wherein the cultured cells are transfected with an KIBRA expression construct previous to the contacting of the cells with the test substance, whereas the expression construct comprises a KIBRA full length cDNA, including 3′ and/or 5′ untranslated regions (UTR), under the control of a exogenous promoter, and whereas the quantification of the expression level of said full length cDNA reflects the alteration of its stability due to the contacting of the cells with the test substance.
11. The method of 3, wherein the level of endogenous KIBRA protein is quantified using anti-KIBRA antibodies within quantitative protein detection methods such as ELISA methods, Western blot methods, or immunoprecipitation methods.
12. The method of 3, wherein the cultured cells are transfected with a KIBRA or KIBRA fusion protein expression construct previous to the contacting of the cells with the test substance, whereas the quantification of the expression level of said KIBRA protein or KIBRA fusion protein reflects the alteration of its stability due to the contacting of the cells with the test substance.
13. The method of 12, wherein the KIBRA fusion protein comprises a tag-peptide or a reporter gene polypeptide linked to the N- or the C-terminus of the KIBRA polypeptide, optionally with a linker sequence between them.
14. The method of 13, wherein the tag-peptide is selected from a group consisting of FLAG-peptide and myc-peptide.
15. The method of any of 5, 6, or 13, the vector of 7 or 8, or the host cell of 9, wherein the reporter gene is selected from the group consisting of a luciferase, a fluorescent protein, beta-galactosidase, chloramphenicol acetyltransferase, beta-glucuronidase, alkaline phosphatase, a resistance-conferring gene, and a gene for growth selection.

1.-14. (canceled)

15. A method for identifying cognition modulating substances, comprising:
a. contacting said substance with cultured cells and
b. assessing the modulation of KIBRA expression within the cells;
wherein the modulation of KIBRA expression on transcript level is assessed;
wherein KIBRA expression is assessed by using cells transfected by a reporter gene construct under the control of a KIBRA promoter.

16. The method of claim 15, wherein the KIBRA promoter is the 3 kb genomic region upstream of the KIBRA coding sequence or any truncated fragment with a length of at least 100 bp or a combination of two or more of these truncated fragments.

17. The method of claim 15, wherein the KIBRA promoter is a homologue nucleic acid which is at least 90% identical to a truncated fragment having a length of at least 500, at least 300, or at least 100 bp of the 3 kb genomic region upstream of the KIBRA coding sequence.

18. The method of claim 15, wherein the KIBRA gene is partly replaced by a reporter construct.

19. The method of claim 15, wherein the cultured cells are transfected with an expression construct prior to the contacting of the cells with the test substance, wherein the expression construct comprises the reporter gene under the control of a KIBRA promoter.

20. The method of claim 19, wherein the expression construct further comprises genomic sequences 3′ to the KIBRA stop codon.

21. The method of claim 20, wherein the KIBRA promoter is the 3 kb genomic region upstream of the KIBRA coding sequence or any truncated fragment with a length of at least 100 bp or a combination of two or more of these truncated fragments.

22. The method of claim 20, wherein the KIBRA promoter is a homologue nucleic acid which is at least 90% identical to a truncated fragment having a length of at least 500, at least 300, or at least 100 bp of the 3 kb genomic region upstream of the KIBRA coding sequence.

23. The method of claim 20, wherein the KIBRA gene is partly replaced by a reporter construct.

24. The method of claim 15, wherein the cells are
i) of a cultured cell line;
or
ii) primary cells.

25. The method of claim 24, wherein the cells are of a cell line that naturally expresses KIBRA.

26. The method of claim 24, wherein the cells are of a kidney or CNS derived cell line.

27. The method of claim 24, wherein the cells are selected from the group consisting of PC12 cells, SH-SY5Y cells, and HEK cells.

28. The method of claim 24, wherein the cells are cells that natively express KIBRA.

29. The method of claim 24, wherein the cells are neural cells or kidney cells.

30. The method of claim 24, wherein the cells are hippocampal cells.

31. An expression vector comprising a reporter gene under the control of a KIBRA promoter.

32. The expression vector of claim 31, wherein the KIBRA promoter is the 3 kb genomic region upstream of the KIBRA coding sequence, or any truncated fragment with a length of at least 100 bp, or homologous thereof having at least 90% identity to said truncated fragments, or a combination of two or more of these truncated fragments.

33. A host cell containing the expression vector of claim 31.

34. The method of claim 15, wherein the reporter gene is selected from the group consisting of a luciferase, a fluorescent protein, beta-galactosidase, chloramphenicol acetyltransferase, beta-glucuronidase, alkaline phosphatase, a resistance-conferring gene, and a gene for growth selection.


SEQUENCE LISTING (13 sequences)

SEQ ID NO: 1 - DNA, 4160 bp, Homo sapiens
  CDS (67)..(3426)
ctggagccgctgagcccccgctgcggccgggagctgcatgggggagcgccggcagcgcttgggaagatgccccggccggagctgcccctgccggagggctgggaggaggcgcgcgacttcgacggcaaggtctactacatagaccacacgaaccgcaccaccagctggatcgacccgcgggacaggtacaccaaaccgctcacctttgctgactgcattagtgatgagttgccgctaggatgggaagaggcatatgacccacaggttggagattacttcatagaccacaacaccaaaaccactcagattgaggatcctcgagtacaatggcggcgggagcaggaacatatgctgaaggattacctggtggtggcccaggaggctctgagtgcacaaaaggagatctaccaggtgaagcagcagcgcctggagcttgcacagcaggagtaccagcaactgcatgccgtctgggagcataagctgggctcccaggtcagcttggtctctggttcatcatccagctccaagtatgaccctgagatcctgaaagctgaaattgccactgcaaaatcccgggtcaacaagctgaagagagagatggttcacctccagcacgagctgcagttcaaagagcgtggctttcagaccctgaagaaaatcgataagaaaatgtctgatgctcagggcagctacaaactggatgaagctcaggctgtcttgagagaaacaaaagccatcaaaaaggctattacctgtggggaaaaggaaaagcaagatctcattaagagccttgccatgttgaaggacggcttccgcactgacagggggtctcactcagacctgtggtccagcagcagctctctggagagttcgagtttcccgctaccgaaacagtacctggatgtgagctcccagacagacatctcgggaagcttcggcatcaacagcaacaatcagttggcagagaaggtcagattgcgccttcgatatgaagaggctaagagaaggatcgccaacctgaagatccagctggccaagcttgacagtgaggcctggcctggggtgctggactcagagagggaccggctgatccttatcaacgagaaggaggagctgctgaaggagatgcgcttcatcagcccccgcaagtggacccagggggaggtggagcagctggagatggcccggaagcggctggaaaaggacctgcaggcagcccgggacacccagagcaaggcgctgacggagaggttaaagttaaacagtaagaggaaccagcttgtgagagaactggaggaagccacccggcaggtggcaactctgcactcccagctgaaaagtctctcaagcagcatgcagtccctgtcctcaggcagcagccccggatccctcacgtccagccggggctccctggttgcatccagcctggactcctccacttcagccagcttcactgacctctactatgacccctttgagcagctggactcagagctgcagagcaaggtggagttcctgctcctggagggggccaccggcttccggccctcaggctgcatcaccaccatccacgaggatgaggtggccaagacccagaaggcagagggaggtggccgcctgcaggctctgcgttccctgtctggcaccccaaagtccatgacctccctatccccacgttcctctctctcctccccctccccaccctgttcccctctcatggctgaccccctcctggctggtgatgccttcctcaactccttggagtttgaagacccggagctgagtgccactctttgtgaactgagccttggtaacagcgcccaggaaagataccggctggaggaaccaggaacggagggcaagcagctgggccaagctgtgaatacggcccaggggtgtggcctgaaagtggcctgtgtctcagccgccgtatcggacgagtcagtggctggagacagtggtgtgtacgaggcttccgtgcagagactgggtgcttcagaagctgctgcatttgacagtgacgaatcggaagcagtgggtgcgacccgaattcagattgccctgaagtatgatgagaagaataagcaatttgcaatattaatcatccagctgagtaacctttctgctctgttgcagcaacaagaccagaaagtgaatatccgcgtggctgtccttccttgctctgaaagcacaacctgcctgttccggacccggcctctggacgcctcagacactctagtgttcaatgaggtgttctgggtatccatgtcctatccagcccttcaccagaagaccttaagagtcgatgtctgtaccaccgacaggagccatctggaagagtgcctgggaggcgcccagatcagcctggcggaggtctgccggtctggggagaggtcgactcgctggtacaaccttctcagctacaaatacttgaagaaacagagcagggagctcaagccagtgggagtcatggcccctgcctcagggcctgccagcacggacgctgtgtctgctctgttggaacagacagcagtggagctggagaagaggcaggagggcaggagcagcacacagacactggaagacagctggaggtatgaggagaccagtgagaatgaggcagtagccgaggaagaggaggaggaggtggaggaggaggagggagaagaggatgttttcaccgagaaagcctcacctgatatggatgggtacccagcattaaaggtggacaaagagaccaacacggagaccccggccccatcccccacagtggtgcgacctaaggaccggagagtgggcaccccgtcccaggggccatttcttcgagggagcaccatcatccgctctaagaccttctccccaggaccccagagccagtacgtgtgccggctgaatcggagtgatagtgacagctccactctgtccaaaaagccaccttttgttcgaaactccctggagcgacgcagcgtccggatgaagcggccgtccccacccccacagccttcctcggtcaagtcgctgcgctccgagcgtctgatccgtacctcgctggacctggagttagacctgcaggcgacaagaacctggcacagccaattgacccaggagatctcggtgctgaaggagctcaaggagcagctggaacaagccaagagccacggggagaaggagctgccacagtggttgcgtgaggacgagcgtttccgcctgctgctgaggatgctggagaagcggcagatggaccgagcggagcacaagggtgagcttcagacagacaagatgatgagggcagctgccaaggatgtgcacaggctccgaggccagagctgtaaggaacccccagaagttcagtctttcagggagaagatggcatttttcacccggcctcggatgaatatcccagctctctctgcagatgacgtctaatcgccagaaaagtatttcctttgttccactgaccaggctgtgaacattgactgtggctaaagttatttatgtggtgttatatgaaggtactgagtcacaagtcctctagtgctcttgttggtttgaagatgaaccgactttttagtttgggtcctactgttgttattaaaaacagaacaaaaacaaaacacacacacacacaaaaacagaaacaaaaaaaaccagcattaaaataataagattgtatagtttgtatatttaggagtgtatttttgggaaagaaaatttaaatgaactaaagcagtattgagttgctgctcttcttaaaatcgtttagattttttttggtttgtacagctccaccttttagaggtcttactgcaataagaagtaatgcctgggggacggtaatcctaataggacgtcccgcacttgtcacagtacagctaatttttcctagttaacatattttgtacaatattaaaaaaatgcacagaaaccattgggggggattcagaggtgcatccacggatcttcttgagctgtgacgtgtttttatgtggctgcccaacgtggagcgggcagtgtgataggctgggtgggctaagcagcctagtctatgtgggtgacaggccacgctggtctcagatgcccagtgaagccactaacatgagtgaggggagggctgtggggaactccattcagttttatctccatcaataaagtggcctttc

SEQ ID NO: 2 - protein, 1119 aa, Homo sapiens
MPRPELPLPEGWEEARDFDGKVYYIDHTNRTTSWIDPRDRYTKPLTFADCISDELPLGWEEAYDPQVGDYFIDHNTKTTQIEDPRVQWRREQEHMLKDYLVVAQEALSAQKEIYQVKQQRLELAQQEYQQLHAVWEHKLGSQVSLVSGSSSSSKYDPEILKAEIATAKSRVNKLKREMVHLQHELQFKERGFQTLKKIDKKMSDAQGSYKLDEAQAVLRETKAIKKAITCGEKEKQDLIKSLAMLKDGFRTDRGSHSDLWSSSSSLESSSFPLPKQYLDVSSQTDISGSFGINSNNQLAEKVRLRLRYEEAKRRIANLKIQLAKLDSEAWPGVLDSERDRLILINEKEELLKEMRFISPRKWTQGEVEQLEMARKRLEKDLQAARDTQSKALTERLKLNSKRNQLVRELEEATRQVATLHSQLKSLSSSMQSLSSGSSPGSLTSSRGSLVASSLDSSTSASFTDLYYDPFEQLDSELQSKVEFLLLEGATGFRPSGCITTIHEDEVAKTQKAEGGGRLQALRSLSGTPKSMTSLSPRSSLSSPSPPCSPLMADPLLAGDAFLNSLEFEDPELSATLCELSLGNSAQERYRLEEPGTEGKQLGQAVNTAQGCGLKVACVSAAVSDESVAGDSGVYEASVQRLGASEAAAFDSDESEAVGATRIQIALKYDEKNKQFAILIIQLSNLSALLQQQDQKVNIRVAVLPCSESTTCLFRTRPLDASDTLVFNEVFWVSMSYPALHQKTLRVDVCTTDRSHLEECLGGAQISLAEVCRSGERSTRWYNLLSYKYLKKQSRELKPVGVMAPASGPASTDAVSALLEQTAVELEKRQEGRSSTQTLEDSWRYEETSENEAVAEEEEEEVEEEEGEEDVFTEKASPDMDGYPALKVDKETNTETPAPSPTVVRPKDRRVGTPSQGPFLRGSTIIRSKTFSPGPQSQYVCRLNRSDSDSSTLSKKPPFVRNSLERRSVRMKRPSPPPQPSSVKSLRSERLIRTSLDLELDLQATRTWHSQLTQEISVLKELKEQLEQAKSHGEKELPQWLREDERFRLLLRMLEKRQMDRAEHKGELQTDKMMRAAAKDVHRLRGQSCKEPPEVQSFREKMAFFTRPRMNIPALSADDV

SEQ ID NO: 3 - DNA, 4467 bp, Mus musculus
  CDS (379)..(3693)
cgcgcggcggggctccggcagaggctgcgggcggcggcggcggcggcggcggcgtggagcgcagcgcgcagcggcaggggcaggcaggcggcggctcgggccgctctcctggctggctggccgcggactcgggccggctgggcgccgcggcgggcgcacatggcagcgtgagaggcgggcggcgtggggctggggccgcagggccgcatggacagaggcgccaccccggccggcccccgttagggcccccgatccgtgggcgccactccccgatcatgctgcctcggcggccgcctggacccaaagcggccagcccgagccgctgagccccgtggcggctgcgagctgcatgggggagcgcgggccaggctcgggaagatgccccggccggagttgcccctgccggagggctgggaggaggcgcgcgacttcgacggcaaggtctactacatagatcacaggaaccgcaccaccagctggatcgacccgcgggacaggtacaccaaaccactcaccttcgctgactgcatcagtgatgagttaccgctgggatgggaagaagcatacgacccacaggtcggagattacttcatagaccacaataccaaaaccactcagatcgaggatccaagggtgcaatggcggcgggaacaggagcacatgctgaaggattacctggtggtggctcaggaagctctgagtgcacaaaaggagatctaccaggtgaagcagcagcgcctggagctggcacagcaggagtatcagcagttgcatgccgtctgggagcataagctgggctcccaggttagcttggtctctggatcctcgtccagctccaagtatgaccccgagatcctgaaagctgaaattgccactgcaaagtcccgggtcaacaagctgaagagagagatggttcacctacagcatgagctgcagttcaaggagcgtggctttcagacgctgaagaaaattgatgagagaatgtcagacgcacagggtggctacaaactggatgaggcccaggctgtgctgagggaaaccaaagccatcaagaaagccatcacctgcggagaaaaggagaaacaagacctcattaagagtctcgccatgctgaaggatggcttccggacagacagagggtcacactccgacctatggtccagcagcagctctctggagagttcaagcttcccgatgccaaagcagttcctggacgtgagctcgcagacagacatctctgggagtttcagcaccagcagcaacaatcagttggccgagaaggtcagattgcgccttcggtatgaagaggcgaagagaaggatcgccaacctcaagatccagctggccaaacttgacagtgaggcctggcctggggtgctagactcagagagggaccgactgatcctcatcaatgagaaggaggaacttctgaaggaaatgcgcttcatcagcccccggaaatggacccagggagaggtggagcaactggagatggcccggaggcgacttgagaaggacttacaggcagcccgggacacccagagcaaggcgctgactgagaggctgaagttaaatagcaagaggaaccagctggtgcgagaactggaggaagccactcggcaggtggctaccctgcactcccagttgaaaagcctctcaagcagcatgcagtccctgtcttcaggcagtagccctgggtcactcacctccagccggggctccctggccgcctctagcctggactcctccacctcagccagcttcactgacctctattatgacccgttcgagcagttggactcggagctccagagcaaggtggagctgctgttcctggagggtgccacgggcttccggccctcaggctgcatcaccaccatccacgaggatgaggtggccaagacccagaaggcagagggaggcagccgcctgcaggccctgcgctccctatctggcactccaaggtccatgacctccctgtctccacgctcatctctctcctccccatcccctccctgttcccctctcatcactgatcccctcctgactggagatgccttcctggccccgctggagtttgaagacacagagctgagtaccactctctgtgagctgaacctcggcggtagcggcacccaggaaagataccggctggaggaaccaggacctgagggcaagccactgggccaagctgcaagtgtggccccaggatgtggcctcaaagtagcctgtgtgtccgctgcagtgtccgacgagtcagtagccggggacagtggcgtgtatgaggcttcagcacagagaccaggtacttctgaagctgccgcattcgacagtgatgaatcggaagctgtgggtgccactcgggttcagattgccctgaaatatgatgagaagaataagcaatttgcaatattaatcattcagctgagtcacctctctgctctgtcactacaacaagaccagaaagtgaacatccgtgtagccatcctgccttgctctgaaagcagcacctgcttgttcagaacccggcctctggactcggcaaataccctcgtgttcaatgaggcattctgggtgtccatctcctacccagcccttcaccaaaagaccttacgagtcgatgtctgcaccactgacaggagccatacagaggagtgtctgggaggtgcccagatcagcctggccgaggtctgccggtctggggagaggtcaacgcgctggtacaatctcctgagctacaaatacttgaagaagcagtgcagggagccccagccaacagaagctccagggccagaccatgtggacgctgtgtctgccttgctagaacagacagccgtggagctagagaagaggcaggagggaagaagcagctcccagacgctggagggcagctggacgtacgaggaagaggccagcgagaatgaggcagtcgctgaggaggaagaggagggagaggaagatgtcttcactgaaaaggtctctcccgaggcagaggagtgcccagcattaaaggtggacagagagaccaatactgacagcgtggccccatcccccacagtggtgcgccctaaggaccggagggtgggtgccccgtcgacagggccatttcttcgagggaataccatcatccgctccaagaccttctcgcctggaccccagagccagtatgtgtgtcggctaaaccgcagtgacagtgatagttctacgctgtccaagaagccaccttttgtgcgaaactccctggagcggcgcagcgtcaggatgaagcggccttcctccgtcaagtccctgcgcacagagcgtctgatccgcacctcgctggacctagaactcgacctgcaggccacgaggacctggcacagccagctgactcaggagatctcagtgctgaaggagctgaaggagcatttggagcaagccaagaaccacggggagaaggagctgccacagtggctgcgtgaagacgagcgcttccgcttgctactgaggatgctggagaagaaggtggatcgtggggagcacaagagtgagttgcaagccgacaagatgatgagagctgctgccaaggatgtgcacaggcttcgaggccagagctgtaaggagcccccagaagtgcagtctttcagggagaaaatggcatttttcacccggcctcggatgaacatcccagctctctctgcagatgacgtctaaccaccagaaaagtatttcctttgtctcgtgcgcgcgcgtgcagaccaagctgtgaaggctgactgtgactaatgttatttatgaggcattatatgaaggtactgagtcacaagtccccgagcgctcttgttggtttgaagacagattttttttttaaatttcaggttttcttgttattatttaaaaaaaaaaatgacaaagcaaccataacaacaaaaacaaaaaccaaacaaacaaaaaaaccagcattaaaatgacaagattgtatagtttgtatatttaggaatgtatttttgggaaagaatttaaatgaactaaagcagcattaagtggctgctagtcttaaaactgtctagagttttctttttgtacagcaacaactttcagaggtttccctgcaataataaaaaaaaaaaaagttgtatttgaggggcgacaatcctcaaaggaggtcccgcgggtggcacagcacagctagcctgccctggttaacgttattttgtacaataatacaaagacaaggatgtacagtaatcgtgggggaattcgaagactgtccgcggatctttgtgagctgtgatgtgtctttcatgtggccacgtgaccgtgaccggaagcaggtagcctggagctggctgaaccgcccggactggtgacaggccacgcccaggccacgcccatctcagatgcccagtgacgcagctgtgggagtgaggggagggctgcggagaaccgcatttggatctatctctaacaataaagtggccttttaag

SEQ ID NO: 4 - protein, 1104 aa, Mus musculus
MPRPELPLPEGWEEARDFDGKVYYIDHRNRTTSWIDPRDRYTKPLTFADCISDELPLGWEEAYDPQVGDYFIDHNTKTTQIEDPRVQWRREQEHMLKDYLVVAQEALSAQKEIYQVKQQRLELAQQEYQQLHAVWEHKLGSQVSLVSGSSSSSKYDPEILKAEIATAKSRVNKLKREMVHLQHELQFKERGFQTLKKIDERMSDAQGGYKLDEAQAVLRETKAIKKAITCGEKEKQDLIKSLAMLKDGFRTDRGSHSDLWSSSSSLESSSFPMPKQFLDVSSQTDISGSFSTSSNNQLAEKVRLRLRYEEAKRRIANLKIQLAKLDSEAWPGVLDSERDRLILINEKEELLKEMRFISPRKWTQGEVEQLEMARRRLEKDLQAARDTQSKALTERLKLNSKRNQLVRELEEATRQVATLHSQLKSLSSSMQSLSSGSSPGSLTSSRGSLAASSLDSSTSASFTDLYYDPFEQLDSELQSKVELLFLEGATGFRPSGCITTIHEDEVAKTQKAEGGSRLQALRSLSGTPRSMTSLSPRSSLSSPSPPCSPLITDPLLTGDAFLAPLEFEDTELSTTLCELNLGGSGTQERYRLEEPGPEGKPLGQAASVAPGCGLKVACVSAAVSDESVAGDSGVYEASAQRPGTSEAAAFDSDESEAVGATRVQIALKYDEKNKQFAILIIQLSHLSALSLQQDQKVNIRVAILPCSESSTCLFRTRPLDSANTLVFNEAFWVSISYPALHQKTLRVDVCTTDRSHTEECLGGAQISLAEVCRSGERSTRWYNLLSYKYLKKQCREPQPTEAPGPDHVDAVSALLEQTAVELEKRQEGRSSSQTLEGSWTYEEEASENEAVAEEEEEGEEDVFTEKVSPEAEECPALKVDRETNTDSVAPSPTVVRPKDRRVGAPSTGPFLRGNTIIRSKTFSPGPQSQYVCRLNRSDSDSSTLSKKPPFVRNSLERRSVRMKRPSSVKSLRTERLIRTSLDLELDLQATRTWHSQLTQEISVLKELKEHLEQAKNHGEKELPQWLREDERFRLLLRMLEKKVDRGEHKSELQADKMMRAAAKDVHRLRGQSCKEPPEVQSFREKMAFFTRPRMNIPALSADDV

SEQ ID NO: 5 - DNA, 4181 bp, Rattus norvegicus
  CDS (93)..(3419)
gcggccgcctggacccaaagcggccggcctgagccgctgagccccgtggcggccgcgagctgcatgggggagcgcgggccaggctcgggaagatgccccggccggagttgcccctgccggagggctgggaggaggcgcgcgacttcgacggcaaggtctactacatagatcacaggaaccgcaccaccagctggatcgacccgcgggacaggtacaccaaaccactcacctttgctgactgcattagcgacgagttgccgctgggatgggaagaagcgtatgatccacaggttggagattacttcatagaccacaataccaaaaccactcagatcgaggatccgagggtgcaatggcggcgggaacaagagcacatgctgaaggattacctggtggtggcccaggaggctctgagtgcacaaaaggagatctaccaggtgaagcagcagcgcctggagctggcacagcaggagtatcagcagttgcatgccgtctgggagcataagctgggctcccaggttagcttggtctcgggatcctcctccagctcaaagtatgaccctgagatcctgaaagctgaaattgccaccgcaaagtcccgggtgaacaaactgaagagagagatggtccacctacagcacgagctacagttcaaggagcgtggctttcagaccctgaagaaaattgatgagaggatgtccgatgctcagggtggctacaaactggacgaagcccaggctgtgctgagggaaaccaaagccatcaagaaagccatcacctgcggagaaaaggagaaacaagacctgatcaagagtctcgccatgctgaaggacggcttccggacggacagagggtcgcactccgacctgtggtccagcagcagctctctggagagttcgagcttcccagtgccaaagcagttcctggatgtgagctcgcagacagacatctctgggagtttcagcaccagcagcagcaaccagttggccgagaaggtccgattgcgccttcggtatgaagaggcaaagagaaggatcgccaacctcaagatccagctggccaaacttgacagtgaggcctggcctggagtgctagactcagagagggaccgactgatcctcatcaatgagaaggaggagcttctgaaggagatgcgcttcatcagcccccggaaatggacgcagggtgaagtggagcagctagagatggcccggaggagactggagaaggacttgcaggcagcccgggacacccagagcaaggcgctgactgagaggttgaagttaaacagcaagaggaaccagctggtgcgagaactggaggaagccacgcggcaggtggccgccctgcactctcagttgaaaagcctctccagcagtatgcaatcactgtcttcaggcagtagccctgggtcacttacgtccagccggggctccctggctgcctctagcctggactcctccacctcagccagcttcactgacctctattacgacccgttcgagcagctggactcagagctcccgagcaaagtagaactgctgttcctggagggtgccacaggcttccggccctcaggctgcatcaccaccatccacgaggatgaggtggccaagactcagaaggcagagggaggcagccgcctgcaggccctgcgctccctatccggcactccaagatccatgacctccctgtctccacgctcatctctctcctccccgtcccctccctgttcccctctcatcgctgaccccctcctggctggagatgcctttctgacccctctggagtttgaagacacagagctgagccccactctttgtgagctgagcctcggcaccgatggcgcccgggaaagattccggctggaggaaccaggacccgagggcaaaccactgggccaggctgcaagtgtggccccagggtgtggcctcaaagtggcctgtgtgtccgctgcagtgtctgatgagtcagtggccggggacagcggcgtgtatgaggcttccatgcagagactaggtacttctgaggctgctgcattcgacagcgacgagtcggaagctgtgggtgtcactcaggttcagattgccctgaaatatgatgagaagagtaagcagtttgcaatattagtcatccagctgagtaacctctctgccctgttactacagcaagatcagaaagtgaacatccgtgtagccatcttgccttgctctgaaagcagcacctgcttgttcagaacccggcctctggactctgcagatagccttgtgttcaatgaggcattctgggtgtccatgtcctacccagcccttcaccagaagaccttacgagttgatgtctgcaccactgacaggagccatacagaggagtgcctgggaggtgcccagatcagcctggctgaggtctgccggtccggggagaggtcaacccgctggtacaaccttctgagctacaaatacttgaagaaacagggcagggagccccagccagcagaagctccagggcccgaccatgtggatgccgtgtctgccttgctagaacagacagctgtggaactagagaagaggcaggaggggagaagcagctcccagacactggagggcagctggacgtacgaggaagaggccagcgagaatgaggcagtagctgaggaggaagagagagaggaagaggagggagaggaagatgtcttcgctgaaaaggtctccccggaggcagaggagtgcccagcattaaaggtggacagagagaccaatactgacagcgtgaccccatcccccacagtggtgcgccccaaagaccggcgggtgggtgccccctcgccagggccatttcttcgagggaataccatcatccgctccaagaccttctcgcctggaccccagagccagtatgtgtgtcggctgaatcgaagcgacagcgacagctccacgctgtccaagaagccaccctttgttcgaaactccctggagcggcgcagcgtcaggatgaagcggccttcgtccgtcaagtcgctgcgcacagagcgcctgatccgcacctcgctggacctagaactagacctgcaggccacgaggacctggcacagccagctgactcaggagatctcagtgctgaaggaactgaaggagcatttggagcaagccaagaaccacggggagaaggagctgccacaatggctgcgtgaagatgagcgcttccgcctcctactgaggatgctggagaagagggtggatcgcggggagcacaagagcgagttgcaggccgacaagatgatgagggcagctgccaaggacgtgcacaggctccgaggccagagctgtaaggagcccccggaagtgcagtctttcagggagaaaatggcatttttcacccggcctcggatgaacatcccagctctctctgccgatgacgtctaaccaccagaaaagtatttcctttcttgcacggaccatgctgtgacggctgactgtgactaaagttatttatgaggcattatgtgaaggtaccgagtcacaggtccccgagcgctctcgttggtttgaagacaaacagatttttttttttagttcaggttttcttgatattatttaaaaaaaaaatgacaaagcaaccataacaacaacaacaaaaacaaacaaacaaacaaacaaaaaaaccagcattaaaatgacaagattgtatagtttgtatatttaggaatgtatttttgggaaagaatttaaatgaactaaagcagcgttaagcggctgctagtcttaaaattgtctagagttttctttttgtacagcaacaacttttagaggtttccctgcaataaaaaaaaaaaaaagaagtcgcatttgaagggcgataatcctccgaggaggtcccgcaggtggcacggcacagctaacctgtcctagttaacattattttgtacaataatacaaagaggaggatgtacagaaatcgtggggttgtcgcagactgtcctcggatctttgtgagcttcgatgtgtcttcgtgtggccacctgacctgaagcaggcagcgtgaagctggctgggcaaggccagcccagacaggcggcaggccacgcccatctcagatacccagtgacactgctgcgggagtgaggggagggccgcagagaactgcatttggatctctcgctatcaataaagtggccttttaagaaa

SEQ ID NO: 6 - protein, 1108 aa, Rattus norvegicus
MPRPELPLPEGWEEARDFDGKVYYIDHRNRTTSWIDPRDRYTKPLTFADCISDELPLGWEEAYDPQVGDYFIDHNTKTTQIEDPRVQWRREQEHMLKDYLVVAQEALSAQKEIYQVKQQRLELAQQEYQQLHAVWEHKLGSQVSLVSGSSSSSKYDPEILKAEIATAKSRVNKLKREMVHLQHELQFKERGFQTLKKIDERMSDAQGGYKLDEAQAVLRETKAIKKAITCGEKEKQDLIKSLAMLKDGFRTDRGSHSDLWSSSSSLESSSFPVPKQFLDVSSQTDISGSFSTSSSNQLAEKVRLRLRYEEAKRRIANLKIQLAKLDSEAWPGVLDSERDRLILINEKEELLKEMRFISPRKWTQGEVEQLEMARRRLEKDLQAARDTQSKALTERLKLNSKRNQLVRELEEATRQVAALHSQLKSLSSSMQSLSSGSSPGSLTSSRGSLAASSLDSSTSASFTDLYYDPFEQLDSELPSKVELLFLEGATGFRPSGCITTIHEDEVAKTQKAEGGSRLQALRSLSGTPRSMTSLSPRSSLSSPSPPCSPLIADPLLAGDAFLTPLEFEDTELSPTLCELSLGTDGARERFRLEEPGPEGKPLGQAASVAPGCGLKVACVSAAVSDESVAGDSGVYEASMQRLGTSEAAAFDSDESEAVGVTQVQIALKYDEKSKQFAILVIQLSNLSALLLQQDQKVNIRVAILPCSESSTCLFRTRPLDSADSLVFNEAFWVSMSYPALHQKTLRVDVCTTDRSHTEECLGGAQISLAEVCRSGERSTRWYNLLSYKYLKKQGREPQPAEAPGPDHVDAVSALLEQTAVELEKRQEGRSSSQTLEGSWTYEEEASENEAVAEEEEREEEEGEEDVFAEKVSPEAEECPALKVDRETNTDSVTPSPTVVRPKDRRVGAPSPGPFLRGNTIIRSKTFSPGPQSQYVCRLNRSDSDSSTLSKKPPFVRNSLERRSVRMKRPSSVKSLRTERLIRTSLDLELDLQATRTWHSQLTQEISVLKELKEHLEQAKNHGEKELPQWLREDERFRLLLRMLEKRVDRGEHKSELQADKMMRAAAKDVHRLRGQSCKEPPEVQSFREKMAFFTRPRMNIPALSADDV

SEQ ID NO: 7 - DNA, 23 bp, Artificial
  Primer for RT-PCR of human KIBRA
caccagaagaccttaagagtcga

SEQ ID NO: 8 - DNA, 22 bp, Artificial
  Primer for RT-PCR of human KIBRA
tcactatcactccgattcagcc

SEQ ID NO: 9 - DNA, 21 bp, Artificial
  Primer for RT-PCR of mouse and rat KIBRA
gaaggagctgaaggagcattt

SEQ ID NO: 10 - DNA, 21 bp, Artificial
  Primer for RT-PCR of mouse and rat KIBRA
cctgaaagactgcacttctgg

SEQ ID NO: 11 - DNA, 3000 bp, Homo sapiens
ttttttcaaatcaaagactgagacttcagtgaagcaagctgcccaaggtcacacagctagaaaaccgggtgggcttgggacccaaacagcctgattctagagctcttgcttctcaaccaaaaaactatgaagcaaacatcaacagagatgaacatttgtcagtatcagtcatgttccacatcccaaccatggggaagttctgaggctgcagagtgatttaaaaaagaaagaaagtctccaatctgggtgacagagcgagactccgtctcaaaaataaaaagaaagaaagaaagaaagtcagccaggcaaggtggctcatgtctgtaagcccagcactttgggaggctgaggtgggcggatcacctgaggtcaggagttcgagaccagcctggccaatatggtgaaactccagctctactgaaaaatacaaaaattagctgggcgtggtggcgcaggcctgtaatcccagctactctggaagctgaggcaggagaattgcttgaatctgggaggcagaggttgtagtgagccgagatcatgccactgcactccagcctgggggatggggggagactccatctcaaaaaaagaaaaaaagaaagtataggttaataactgagaacactgcactagagtcagacctagtctgtcttatgagctctgtatcccaggccaacaacaaagcatcttggagcctcatctgtaaaatgggtataataaatagactttcctatgccagagggttgttatgaagattgagatcatgtattcaaagaacctagcaccaggttggctttgctttggggctactgctctaatcaatagtgggtttttatgtagtaaaggcttgacccaaacagaggaagtgtagagggctgtgggaattcccccataaaggaatggggaattccaacttttgggtgttgagggccagtctgaggctggtgtccctggactctggtgcctggtatttatggaatgaaggagggccaagcactgagccccgaggcctggcctcagggaaggaattaactatgaagaaggcatttggaggcgaggctcactctggtgggtcttttctgtcaagaggagatgagaccacaaacatgattccctctctggctcagcaacaccctggtccatacagcacagggtgctggttcttctggaagaccgtggggctcaggcaagagaagacacccaactgggcttcattcgaccagtccagcttcctgccatatgccgagggcaaaaaagggttaaattcccactcagtacacagatgtaagaggagatggggaagtgaacagccagttgatcattcactcaatcatgctagcttgctttttgtatttaatgctacattttaaatttatttatttatttatttgagacagagtctcgctttgttgcccaggctggagtgcagtggtgcgatcttggctcactgcaacctccacctcccaggttgaagtgattctcatgcctcagcctcccgagtagctgggactacgggcgtgcaccaccacgcccagctaatttattattattattttttagtagagatggggttcactatgttggccaggctggtctgaaatcctgacctcaggtaatctgtctgcctcaccctcccaaagtgctgggattacaggcgtgagccaccgcacccactaattctacatttttaatttctcaagaaacattgtgtctctgattgttttattctcttgaacatttttcaagatggaattttgctttgcttactttcttctttttcatagccccacttctcccaaatccataacacacctacctcaaagtatcacaggaaagaaagcctgtgaagctgctagccacagcctgtcacaagtaagtcctcaatttagctaacaataacaatggcattactattacttttacaaagtgttttccctgacaccctctggaagtcctcccaggaaagcggatccacagcgctcctaattttagggaccctgaggggagccagctacatttccaaatggtggggaagggtggagggtgggcgaaacgtcgtagggagagttgaatagaaggagacagatggtgcctgtgttgggggaagggaccagagatcctggaaaacttatgtttcccaattcgcaagaatgtttccatttaaccacttcttgcctgactcctgattctacaccacagaaaagcagggagtttttaagatttggggccacctgtgccgagagcctggctactccgggtggagagagacaggagcgttttaagtacaggcagagcggtttttggcccagccccctaggtctgggacgcaggtgaggggcggtctctccttcccctggcaggggcagcgtaagggtggccggcggctgggactgtcccacgagggggcgcgtctcgggccctaggacccgccctcgaggcgctgcgcgcggggaggggacccgggaacccaggcgcccggcgctccgcctcccgtttccggggcagcgcggttacctgcaccgcccgagccgcacctggcggaggcagaagtcacaaacccggcgagctcagagcggtggagcggagaggagggacggcacccggctgcagggaggagggaggcggcagcggcagcggcggcgtggagggcgcagcgcgccgcgcggcggaggagggcagacggcggcggcggcggcgctcggctcgctctgcccggccggctgggcgcgcacccgggctcgcaccgcgccgctgcggacgcacatggcagcgtgagaggccggcggcgggaggagcgggcggcgccgggtcggggctgcagggccgcatggacagcggcgccaccccggccggcccctactagggccccccatctgcgggcgccaccccccggatcatggtgcctcggcggccgcccgggctaagagcggccggctggagccgctgagcccccgctgcggccgggagctgcatgggggagcgccggcagcgcttgggaag

SEQ ID NO: 12 - DNA, 3000 bp, Mus musculus
tcagtttggattcctgtggttcagtcacctgcagaataacaaccaactcaacctatcataaggctcagcagcttttacctttggcgtgcttgccctgggcctctgaatgcctagagatgattcacccggcattgcaggggggcaggccaccatctgtagattacatcaccctgcttgcagcttctgggagataacctgggatcctaaggcctaagctttaaggaaccaattgctgttccaaggcatggtctaaaggtatagaggaacaagccactggaaaatgccaagcggtgggcatctggcatacaacacattcatccaggaagcttggaaaataaggcacacacacacacacacacacacacacacaccctagttggatgtgtcataagcagacaagcgtacttttcaggaaggaaaagacatgggttgtctgtgttttttagcagaaaatagttcagttgagccacactttataaaagtgtgctggtcatcagatcagccagggtgctccacgtggaccccttggtgatagcttaatcccttatttttctcatttctttctttcttttttttttttttttttttaattttttgagacagggtttctctgtgttcccctggctgtcctggaactcactctgtagaccaggctggcttcgaactcagaaatccacctgcctctgcctcccaagtgctgggattaaaggcgtgcgccatgatgccctgcttctcatttcattttgctcttcccaaccaggtggagggactcttcattatcacatttttctcaagggagaggcaggaggatgctgagctcaaggccaccctggactgcatagcaagaatctaaaaaaacattacaaaccctcgatctaaatacaatgaagcattaatggttgacccttcaaccattggtttcaccggtgctagggtgcttgtgtctcttcttagctccatccctggccatcttgaagttcagtgtcctcatctccagaacgaggagaagtacagtaccctatccagggctttgtcgtgaagattagctgagatcatacttgaaaacgcaagcctcgtatcagatcagcacgtgttggaagtgatggttcgttcacacttgtgatctcacacttgggaggcagaggtaggaaggggagataaccatagtataaggccaggcagagcgaggtctcccaggcaaaaactcaaaacagattggctggtttggttcagtgaggatgcatgcctggagtttgatcctaacagaacgggggaaggagggcactagaagtacttataattgtgaaggctttgcccatcacaggaagtctggagggagaactccccagcagttgggtgggtggtgaggacctccctgagatctgagatccctggcttcgaagtataatattgatgggatggaggaggagctacacccgtcccctatgtcctcagggcatttagatttgaggagcccaccctaaaacgccttgtgtatcaaacaggggatccccacacagtacagggagttaactggttactccccacattaagggattgattcagacaaggcgctggacttcgttcttccagtccagtttcctgcctcaggcacagttacatttgcactgggggaaacaccaaacctaagagggaactttttccccttaattcaatattgctaatgtctcaagacatttacttattcttgtttcagggtttgccttttcttttctcctttcttttctccttccttccttccttccttccttccttccttccttccttccttccttccttccttccttcctccctctctctctctttctttctttctttctttcttttccttctctctctttctctctctctctctctctctctctctctctctctctctctctctctccctctccccctccctttccttccttccttatttatttatttattccccccccccgtgtttgctagtttgactcagcctggaacttttggatttaagtaatccttcttcagcctcttgagtagccgagagacaggtgcacttcactatgactggcaaaatatgtccttccttctcctcccttgccacccgctggaagtcctcttaggaagcagatccatagcattcctaattttagggaccctaaggaaagccagctacatttgcaaattacggagaggagggttgaaacaacacggggagaactgaataggaggaagacagatggtgtgagtgctgggggggaagggacgagagatcctggaaaacttatgttcgccgatttgcaagaatgttttcacttaaccacttcttgcctgactcctggttctcaatcacagaaaacagggtgtgtcaagatttggggccacacgcgccaagagcccggacactctggcgtagagagagcctagcctcctggggcagaacggggtggtcggcgattgggactgtcccacgagggggcgcgtctcgggctctaggacccgccctcgccgcgctgggcgcggggaggagacccgggaaccctggagcccggcgctccgcctcccgtttccggggcagcgcagttacctgcaccgccggagccacacctggcggaggcagaagtctcggggcgcgcggcggggctccggcagaggctgcgggcggcggcggcggcggcggcggcgtggagcgcagcgcgcagcggcaggggcaggcaggcggcggctcgggccgctctcctggctggctggccgcggactcgggccggctgggcgccgcggcgggcgcacatggcagcgtgagaggcgggcggcgtggggctggggccgcagggccgcatggacagaggcgccaccccggccggcccccgttagggcccccgatccgtgggcgccactccccgatcatgctgcctcggcggccgcctggacccaaagcggccagcccgagccgctgagccccgtggcggctgcgagctgcatgggggagcgcgggccaggctcgggaag

SEQ ID NO: 13 - DNA, 3000 bp, Rattus norvegicus
ctctctgcctccgtctctccatcccctgaacgtttactttattgcctttttttattgcgtgtctatgaacatgggatcgccggtaccttcaggccagaggtgtcagatcccctggagcaagagatataggcagttacgagctgccagatgtgggggctgggaagtggaatccacgttctattagaaagtggtacatgctcttacctggtgagcatccccccaccaccacagcaccatcccccaccccgccattcaatttctcctcctcattctcatgaaatattgccccatctcttaagacaggtgtggaaagtggcctttctttgttgttgcagtgcacactgactttaagaccatggatggggctggggatttagctcagtggtagagcgcttacctaggaagcgcaaggccctgggttcggtccccagctccggggggaaaaaaagaaccaaaaaaaaaaaaaaaaaaaaaagaccatggccacctgtggcgttacctccagcaagctacctaaccttgagtgcaaatgcagacttggtgtggttggtagtctctgctcgatcaaccctgacgatatccgcactctcattttgtaagccagagagccctaagaggaaggccgggaggctgcttgccttcagcttggattcctgtggtccagtcacctgtagaataacaaccaattcaacctatcacaaggctcagcaggttttacctttggcatgcttgcccggggcctctgaatgcctggagatgattcaccaggcattgcaggggggcaggccaccatctgtagattacatcaccctgctttcggcttctgggagataacctgggatcctaaggcctaagctttaaggaatggatttctcttccaaggcatagtctaaaggtataaaggaacaagacactggaaaatgccaggggtaacacactcacccaaaaagcttggaaaataaggtacacacacacacacacacacacacacacacactaactggacgtgtcataagcagccaaacatacttttcaggaagaaaaagacatgggttatttgtttgtttttagcagaaactagttcagttggtgatggcttaagcccttatttttctcacttcgtttggcttttcccaatcagttggagggactcttacgttactatattcttctcagagggagaggcgggaggatgttgagcttgaggccaccctagcctgcatagcaagaatctaaaaaatattcggaacccagatgtagatacaatgaaggattgatggttgcccaaggcaaaaactcaaaacaattgaggacactcacttggcaagtgtgacacctggggtttgatccctgacagcacaggaaggggatgtgaaggctttgacccatcagaggaagtctagaggaagagagaaccctcccactcttgggtggtgaggagctccctaggatctgagatccctggctttgaagctaatattgatggactggaggaagaactatactcacccccgatgtccccagggcattaagacttggggagcccaccctaaaacgccttgtctagcaaaaggagctgaagcccaaacaggggattcccacacagtacagggagttgactggttactccccaaatgaagagattctgacaaggcactggacttcattcttctagtccagcttcctgcctcaggcatagttaaatttgcactggggaaacaccagacctaagagtgaacaggcagagggcaaagccagaggattcttggctggatgacaccatcttcttttcccccttgattcagtattgttagtgtctcgaggcatttacttacattcttgttttttggtttgccttttctttttcttcttttttcctttctttctttcttccttttttcttttctttctttctttccttctttctttctttctttccttcttccttactattaatttatttatcccctgtgtttgctattttgacgtggcctggaacttttagatttaagcaatctttcttcctcagcctcttgagtagctgataggcaggtgcacttcactatgactggcaaaatatgtccttccttctcctcccttgccacccgctggaagtcctcttagggaagcagatccattgcattcctaattttagggaccccaaggaaagccagccacatttgcaaattacggaggggagggttgaaacaacacggggagaattgaataggagggagacagatggtgtgagtgctgggggaagggacgagagatcctggaaaacttatgttcgccgatttgcaagaatgttttcacttaaccacttcttgcctgactcctgagtctatctcacagaaaacagggtgtgtcaagatttggggccacatgcaccaagagcctggacattcggtgtagagagagtctagcctcctggggcagagcaggggtcgtcggcgattgggactgtcccacgagggggcgcgtctcgggctctaggacccgccctcgccgcgctgggcgcggggaggagacccgggaaccccggagcccggcgctccgcctcccgtttccggggcagcgcagttacctgcaccgccggagccacacctggcggaggcagaagtctcggggcgcgcaacggggcgccggcagtggctgcgagaggcagcggcggcgtggagcgcagcgcggggcggctggggcaggcaggcggcggctcgggccgctctcccggctggctggccgcgtactcgggccggctgggcgccgcggcgggcgcacatggcagcgtgagaggcgggcggcgcggggctggggccgcagggccgcatggacagaggcgccactccggccggcccccgttagggcccccgatccgcgggcgccaccccccgatcatgctgcctcggcggccgcctggacccaaagcggccggcctgagccgctgagccccgtggcggccgcgagctgcatgggggagcgcgggccaggctcgggaag